SEQUENCE LISTING (8 sequences)

SEQ ID NO: 1 - protein, 298 aa, Streptomyces albidoflavus
APAAVPAHAVTGYWQNFNNGATVQTLADVPDAYDIIAVSFADATANAGEITFTLDSVGLGGYTDEQFRADLAAKQADGKSVIISVGGEKGAVAVNDSASAQRFADSTYALMEEYGFDGVDIDLENGLNSTYMTEALTKLHEKAGDGLVLTMAPQTIDMQSPENEYFKTALVTKDFLTAVNMQYYNSGSMLGCDGQVYAQGTVDFLTALACIQLENGLDASQVGIGVPASPKAAGGGYVEPSVVNDALDCLTRGTGCGSFKPEKTYPALRGAMTWSTNWDADTGNAWSNVVGPHVDDLP

SEQ ID NO: 2 - DNA, 1294 bp, Streptomyces albidoflavus
gcggccgctccgggcggacgaccgtacggactcctcggccgacccctgcgggaacccttgacaaccccattggtctggaccagtttggtgcccatcgcggtggccaccgtgcgccaactccccgccccctcccgggtggcgggccccgtcggcgcgtccccccacgtccgtgactccccccaccggaggcagcagtggtacgcacctacccccttccgcaccccggccggcgcccctccacgcccggcctccaccgcaggggccggctgaccgccgccctcaccgcggccgtcctcggcgcctccgggctcgccctcaccggccccgcgaccgccggcgagggggcccccgccgcccaggccgccccggccgccgtaccggcccacgcggtgaccggttactggcagaacttcaacaacggcgcgaccgtgcagaccctcgccgacgtgccggacgcctacgacatcatcgccgtctccttcgccgacgccacggccaacgcgggcgagatcaccttcaccctcgactcggtcgggctcggcggctacaccgacgagcagttccgcgccgacctcgccgccaagcaggccgacggcaagtcggtgatcatctcggtcggcggcgagaagggcgcggtcgccgtcaacgacagcgcctccgcccagcgcttcgccgacagcacctacgcgctgatggaggagtacggcttcgacggcgtcgacatcgacctggagaacggcctcaactccacctacatgaccgaggccctcaccaagctccacgagaaggccggggacggcctggtcctcaccatggcgccgcagaccatcgacatgcagtcgcccgagaacgagtacttcaagacggcgctggtcacgaaagacttcctgaccgccgtcaacatgcagtactacaacagcggctcgatgctcggctgcgacggccaggtctacgcgcagggcaccgtcgacttcctcaccgcgctcgcctgcatccagctggagaacggtctcgacgcctcccaggtcggcatcggtgtccccgcctccccgaaggcggccggcggcggctacgtcgagccctccgtggtcaacgacgcgctggactgcctgacccggggcaccggttgtggctcgttcaagccggagaagacctacccggcgctgcgtggcgccatgacctggtcgaccaactgggacgccgacaccggcaacgcctggtcgaacgtggtcggcccgcacgtcgacgacctgccgtaaccccggagccgggcacccgtccgcttcccccgcac

SEQ ID NO: 3 - protein, 376 aa, Streptomyces albidoflavus
GPGPGPREKINLGYFTEWGVYGRNYHVKNLVTSGSAEKITHINYSFGNVQGGKCTIGDSFAAYDKAYTAAESVDGVADTWDQPLRGNFNQLRKLKAKYPHIKVLWSFGGWTWSGGFTDAVKNPAAFAKSCHDLVEDPRWADVFDGIDLDWEYPNACGLSCDSSGPAALKNMVQAMRAQFGTDLVTAAITADASSGGKLDAADYAGAAQYFDWYNVMTYDFFGAWDKTGPTAPHSALNSYSGIPKADFHSAAAIAKLKAKGVPASKLLLGIGFYGRGWTGVTQDAPGGTATGPATGTYEAGIEDYKVLKNTCPATGTVGGTAYAKCGSNWWSYDTPATIKTKMTWAKDQGLGGAFFWEFSGDTAGGELVSAMDSGLR

SEQ ID NO: 4 - DNA, 2712 bp, Streptomyces albidoflavus
gtcgactggtacaacgtgatgacctacgactacttcggcacctgggccgcccagggcccgacggcgccccactcgccgctcaccgcctacccgggcatccagggcgagcacaacacctcctcggccaccatcgccaagctgcggggcaagggcatcccggcgaagaagctgctgctgggcatcggcgcctacggccgcggctggaccggcgtcacccaggacgcccccggcggcaccgccaccggcccggccgccggcacctacgaggcgggcaacgaggagtaccgggtgctggccgagaagtgcccggccaccggcaccgccggcggcaccgcgtacgccaagtgcggcgacgactggtggagttacgacacccctgagacggtgacgggcaagatggcctgggcgaagaagcagaagctcggcggtgccttcctctgggagttcgccggcgacggcgccaagggcgatctgttcagggcgatgcacgaggggctgcgctgaccggccgggcactcacccggaactgacccttcccgcacggccgtccgccgtggcaccggagctccggtcgccgcggcgggcggccgtgtccgcatgtcgccacccccgcgcaccaggcgcgatccggccgaactttcctttggtccagacctcttgacctctggtccagaccttttctactctcgccccactgcggtgggcacatcggtcgtcggtgctcacgggcgtcgcagggttccgcccccatacgtccggacctcttgaggagtacgccttgagtacggtttcccccagcaccgacggcgcccgcagccgtcccagacccctcagccgcttccgccggcgcgcgctggccgcgctcgtcggcctcgcggtccccttcgccgggatggtcggcctcgccgcccccacccaggccgccgaggccgcggccgaccccagcgcctcctacaccaggacgcaggactggggcagcggcttcgagggcaagtggacggtgaagaacaccggcaccgcccccctcagcggctggaccctggagtgggacttccccgccggaaccaaggtgacctcggcctgggacgccgacgtcaccaacaacggcgaccactggaccgccaagaacaagagctgggcggggagcctcgcccccggcgcctcggtcagcttcggcttcaacggcaccggccccggcaccccctcgggctgcaagctcaacggcgcctcctgcgacggcggcagcgtccccggcgacaccccgcccaccgcccccggcacccccaccgccagtgacctcaccaagaactcggtgaagctctcctggaaggcggccaccgacgacaagggcgtcaagaactacgacgtcctgcgcgacggcgccaaggtcgccaccgtcaccgccaccaccttcaccgaccagaacctcgcccccggcaccgactactcctactcggtccaggcccgcgacaccgccgaccagaccggcccggtcagcgcccccgtcaaggtcaccacccccggcgacggcacgggccccggccccggcccccgcgagaagatcaacctcggctacttcaccgagtggggcgtctacggccgcaactaccacgtcaaaaacctggtgacctccggctccgccgagaagatcacccacatcaactactccttcggcaacgtccagggcggcaagtgcaccatcggtgacagcttcgccgcctacgacaaggcgtacaccgccgccgagtcggtcgacggcgtcgccgacacctgggaccagccgctgcgcggcaacttcaaccagctccgcaagctcaaggccaagtacccgcacatcaaggtcctctggtccttcggcggctggacctggtccggcggcttcaccgacgccgtgaagaacccggccgccttcgccaagtcctgccacgacctggtcgaggacccgcgctgggccgacgtcttcgacggcatcgacctcgactgggagtacccgaacgcctgcggcctcagctgcgacagctccggtccggccgcgctgaagaacatggtccaggcgatgcgcgcccagttcggcaccgacctggtcaccgccgccatcaccgccgacgccagctccggcggcaagctcgacgccgccgactacgcgggcgccgcccagtacttcgactggtacaacgtgatgacgtacgacttcttcggcgcctgggacaagaccggcccgaccgcgccccactcggccctgaactcctacagcggcatccccaaggccgacttccactcggccgccgccatcgccaagctcaaggcgaagggcgtcccggcgagcaagctcctgctcggcatcggcttctacggccgcggctggaccggcgtcacccaggacgccccgggcggcaccgccaccggcccggccaccggcacctacgaggcgggcatcgaggactacaaggtcctcaagaacacctgccccgccaccggcaccgtcggcggcaccgcgtacgccaagtgcggcagcaactggtggagctacgacaccccggccaccatcaagaccaagatgacctgggccaaggaccagggcctcggcggcgccttcttctgggagttcagcggtgacaccgcgggcggcgaactggtctccgcgatggactccggcctccgctagccccggaccggcaccccgcccgaaccactagcacgacctcccccgga

SEQ ID NO: 5 - DNA, 905 bp, Streptomyces albidoflavus
atggccgccgtaccggcccacgcggtgaccggttactggcagaacttcaacaacggcgcgaccgtgcagaccctcgccgacgtgccggacgcctacgacatcatcgccgtctccttcgccgacgccacggccaacgcgggcgagatcaccttcaccctcgactcggtcgggctcggcggctacaccgacgagcagttccgcgccgacctcgccgccaagcaggccgacggcaagtcggtgatcatctcggtcggcggcgagaagggcgcggtcgccgtcaacgacagcgcctccgcccagcgcttcgccgacagcacctacgcgctgatggaggagtacggcttcgacggcgtcgacatcgacctggagaacggcctcaactccacctacatgaccgaggccctcaccaagctccacgagaaggccggggacggcctggtcctcaccatggcgccgcagaccatcgacatgcagtcgcccgagaacgagtacttcaagacggcgctggtcacgaaagacttcctgaccgccgtcaacatgcagtactacaacagcggctcgatgctcggctgcgacggccaggtctacgcgcagggcaccgtcgacttcctcaccgcgctcgcctgcatccagctggagaacggtctcgacgcctcccaggtcggcatcggtgtccccgcctccccgaaggcggccggcggcggctacgtcgagccctccgtggtcaacgacgcgctggactgcctgacccggggcaccggttgtggctcgttcaagccggagaagacctacccggcgctgcgtggcgccatgacctggtcgaccaactgggacgccgacaccggcaacgcctggtcgaacgtggtcggcccgcacgtcgacgacctgccgtaaccccggagccg

SEQ ID NO: 6 - DNA, 1107 bp, Streptomyces albidoflavus
atgggctacttcaccgagtggggcgtctacggccgcaactaccacgtcaaaaacctggtgacctccggctccgccgagaagatcacccacatcaactactccttcggcaacgtccagggcggcaagtgcaccatcggtgacagcttcgccgcctacgacaaggcgtacaccgccgccgagtcggtcgacggcgtcgccgacacctgggaccagccgctgcgcggcaacttcaaccagctccgcaagctcaaggccaagtacccgcacatcaaggtcctctggtccttcggcggctggacctggtccggcggcttcaccgacgccgtgaagaacccggccgccttcgccaagtcctgccacgacctggtcgaggacccgcgctgggccgacgtcttcgacggcatcgacctcgactgggagtacccgaacgcctgcggcctcagctgcgacagctccggtccggccgcgctgaagaacatggtccaggcgatgcgcgcccagttcggcaccgacctggtcaccgccgccatcaccgccgacgccagctccggcggcaagctcgacgccgccgactacgcgggcgccgcccagtacttcgactggtacaacgtgatgacgtacgacttcttcggcgcctgggacaagaccggcccgaccgcgccccactcggccctgaactcctacagcggcatccccaaggccgacttccactcggccgccgccatcgccaagctcaaggcgaagggcgtcccggcgagcaagctcctgctcggcatcggcttctacggccgcggctggaccggcgtcacccaggacgccccgggcggcaccgccaccggcccggccaccggcacctacgaggcgggcatcgaggactacaaggtcctcaagaacacctgccccgccaccggcaccgtcggcggcaccgcgtacgccaagtgcggcagcaactggtggagctacgacaccccggccaccatcaagaccaagatgacctgggccaaggaccagggcctcggcggcgccttcttctgggagttcagcggtgacaccgcgggcggcgaactggtctccgcgatggactccggcctccgctagtctagacgg

SEQ ID NO: 7 - DNA, 905 bp, Streptomyces albidoflavus
atggccgccgtaccggcccacgcggtgaccggttactggcagaacttcaacaacggcgcgaccgtgcagaccctcgccgacgtgccggacgcctacgacatcatcgccgtctccttcgccgacgccacggccaacgcgggcgagatcaccttcaccctcgactcggccgggctcggcggctacaccgacgagcagttccgcgccgacctcgccgccaagcaggccgacggcaagtcggtgatcatctcggtcggcggcgagaagggcgcggtcgccgtcaacgacagcgcctccgcccagcgcttcgccgacagcacctacgcgctgatggaggagtacggcttcgacggcgtcgacatcgacctggagaacggcctcaactccacctacatgaccgaggccctcaccaagctccacgagaaggccggggacgggctggtcctcaccatggcgccgcagaccatcgacatgcagtcgcccgagaacgagtacttcaagacggcgctggccacgaaagactttctgaccgtcgtcaacatgcagtactacaacagcggttcgatgctcggctgcaacggccaggtctacgcgcagggcaccgtcgaattcctcaccgcgctcgcctgcatccaactggagaacggtctcgacgcctcccaggtcggcatcggcgtccccgcctccccgaaggcgggcggcggcggctacttcgagccctccgtggtcaacgacccctggactgccttgacccggggcaccggttgtggctcgttcaagccggagaagacctacccggcgctgcgtggcgccatgacctggtcgaccaactgggacgccgacaccggcaacgcctggtcgaacgtggtcggcccgcacgtcgacgacctgccgtaatctagacggat

SEQ ID NO: 8 - DNA, 1104 bp, Streptomyces albidoflavus
atgggctacttcaccgagtggggcgtctacggccgcaactaccacgtcaaaaacctggtgacctccggctccgccgagaagatcacccacatcaactactccttcggcaacgtccagggcggcaagtgcaccatcggtgacagcttcgccgcctacgacaaggcgtacaccgccgccgagtcggtcgacggcgtcgccgacacctgggaccagccgctgcgcggcaacttcaaccagctccgcaagctcaaggccaagtacccgcacatcaaggtcctctggtccttcggcggctggactggtccggcggcttcaccgagccgtgaagaacccggccgccttcgccaagtcctgccacgacctggtcgagcgacccgcgctgggccgacgtcttcgacggcatcgacctcgactgggagtacccgaacgcctgcggcctcagctgcgacagctccggtccggccgcgctgaagaacatggtccaggcgatgcgcgcccagttcggcaccgacctggtcaccgccgccatcaccgccgacgccagctccggcggcaagctcgacgccgccgactacgcgggcgccgcccagtacttcgactggtacaacgtgatgacgtacgacttcttccgcgcctgggacaagaccggcccgaccgcgccccactcggccctgaaattctacagcggcatccccaaggccgaattccaatcggccgcgccatcgccaagctcaaggcgaagggcgtcccggcgagcaagctcctgctcggcatcggcttctacggccgcggctggaccggcgtcacccaggacgccccgggcggcaccgccaccggcccggccaccggcacctacgaggcgggcatcgaggactacaaggtcctcaagaacacctgccccgccaccggcaccgtcggcggcaccgcgtacgccaagtgcggcagcaactggtggagctacgacaccccggccaccatcaagaccaagatgacctgggccaaggaccagggcctcggcggcgccttcttctgggagttcagcggtgacaccgcgggcggcgaactggtctccgcgatgactccggcctccgctagtctagacgg

(19) United States
(12) Patent Application Publication
(10) Pub. No.: US 2002/0069431 A1
(43) Pub. Date: Jun. 6, 2002

(54) Effect of endochitinase and chitobiosidase and their encoding genes on plant growth and development

(76) Inventors: Roxanne M. Broadway, Grass Valley, CA (US); Carmenza E. Gongora, La Francia (CO)

Correspondence Address: Michael L. Goldman, Esq., NIXON PEABODY LLP, Clinton Square, P.O. Box 31051, Rochester, NY, 14603, US

(21) Appl. No.: 09/748,033

(22) Filed: Dec. 22, 2000

Related U.S. Application Data
(60) Provisional application No. 60/172,003, filed on Dec. 23, 1999.

(51) Int. Cl. C12N 15/82

(52) U.S. Cl. 800/290; 800/288; 435/200

(57) ABSTRACT

The present invention relates to enzymes with chitobiosidase and endochitinolytic activity and the genes encoding these enzymes. The chitinolytic enzymes of the invention can be applied to a plant or plant seed under conditions effective to promote plant growth, decrease plant size, and increase plant yield. Alternatively, transgenic plants or transgenic plant seeds transformed with a DNA molecule encoding a chitinolytic enzyme can be provided and the transgenic plants or plants resulting from the transgenic plant seeds are grown under conditions effective to promote plant growth, decrease plant size and increase plant yield.

The present application claims benefit the benefit of U.S. Provisional Patent Application Ser. No. 60/172,003, filed Dec. 23, 1999.

### FIELD OF THE INVENTION

The present invention is directed to methods of promoting plant growth and effecting development by transgenic insertion of endochitinase and chitobiosidase genes.

### BACKGROUND OF THE INVENTION

Chitin, an insoluble linear β-1,4-linked polymer of N-acetyl-β-D-glucosamine, is a structural polysaccharide that is present in all arthropods, yeast, most fungi, and some stages of nematodes. Chitinolytic enzymes are proteins that catalyze the hydrolysis of chitin by cleaving the bond between the C1 and C4 of two consecutive N-acetylglucosamines. There are three types of chitinolytic enzyme activity: (1) N-acetyl-β-glucosaminidase (i.e., EC 3.2.1.30, abbreviated glucosaminidase), which cleaves monomeric units from the terminal end of chitin; (2) 1,4-β-chitobiosidase (i.e., abbreviated chitobiosidase), which cleaves dimeric units from the terminal end of chitin, and (3) endochitinase (EC 3.2.1.14), which randomly cleaves the chitin molecule internally (Sahai et al., “Chitinases of Fungi and Plants: Their Involvement in Morphogenesis and Host-Parasite Interaction,” FEMS Microbiol. Rev., 11:317-38 (1993)). Two or three types of enzymes are often synthesized by a single organism (Harman et al., “Chitinolytic Enzymes of Trichoderma harzianum: Purification of Chitobiase and Endochitinase,” Phytopathology, 83:313-18 (1993), Neugebauer et al., “Chitinolytic Properties of Streptomyces lividans,” Arch. Microbiol, 156:192-97 (1991), Romaguera, et al., “Protoplast Formation by a Mycolase from Streptomyces olivaceoviridis and Purification of Chitinases,” Enzyme Microb. Technol., 15:412-17 (1993), which are hereby incorporated by reference), which may enhance the speed and/or efficiency of degradation of chitin.

Plant chitinolytic enzymes and β-1-3 glucanases are among a group of proteins that are inducible in plants in response to various forms of stress, and are generally believed to serve protective functions in the plants, although the exact nature of those functions is not clear (Boller, “Hydrolytic Enzymes in Plant Disease Resistance,” in Kosuge T. and Nestor E, eds., Plant-Microbe Interaction, New York: Macmillan, pp. 385-480 (1987)). Chitinolytic enzymes have a role as phytochemical defense agents against pathogenic fungi as indicated by (1) the coordinated induction of those enzymes in response to pathogen invasion (Roby et al., “Induction of Chitinases and of Translatable mRNA for these Enzymes in Melon Plants Infected with Colletotrichum lagenarium,” Plant Sci., 52:175-185 (1987)), (2) the fact that chitinolytic enzymes from plants are potent inhibitors of fungal spores germination and mycelial growth in vitro (as demonstrated by their ability to hydrolyze fungal cell wall) (Broekaert et al.,. “Comparison of Some Molecular, Enzymatic and Antifungal Properties of Chitinases from Thorn-Apple, Tobacco and Wheat,” Physiol. Molecu. Plant Pathol. 33:319-331 (1988); and Schlumbaum et al., “Plant Chitinases are Potent Inhibitors of Fungal Growth,” Nature 324:365-367 (1986)), (3) higher levels of chitinolytic activity are observed in resistant cultivars compared with susceptible cultivars (Hughes et al., “Modulation of Elicitor-Induced Chitinase and β-1,3-Glucanase Activity by Hormones in Phaseolus Vulgaris,” Plant Cell Physiol. 32:853-861 (1991); and Vogelsang et al., “Elicitation of β-1,3-Glucanase and Chitinase Activities in Cell Suspension Cultures of Ascochyta rabiei Resistant and Susceptible Cultivars of Chickpea (Cicer arietinum),” Z. Naturforsch 45c:233-239 (1990)) and, (4) enhance resistance of plants following transformation of the plants with chitinolytic enzymes (Broglie et al., “Transgenic Plants with Enhance Resistance to the Fungal Pathogen Rhizoctonia solani,” Science 245:1194-1197 (1991); Grison et al., “Field Tolerance to Fungal Pathogens of Brassica napus Constitutively Expressing a Chimeric Chitinase Gene,” Nature Biotechnology 14:643-646 (1996); and Tabei et al., “Transgenic Cucumber Plants Harboring a Rice Chitinase Gene Exhibit Enhance Resistance to Gray Mold (Botrytis cinerea),” Plant Cell Report 17:159-164 (1998))

However, among the different chitinase isoforms produced in plants, not all of them have antifungal properties. Schickler et al., “Heterologous Chitinase Gene Expression to Improve Plant Defense Against Phytopathogenic Fungi,” Journal of Industrial Microbiology & Biotechnology 19:196-201 (1997) indicated that the phenomenon of variable antifungal potency chitinases is problematic. The success of the defense mechanism depends on both the type of chitinase and the species of fungus. Not all plants that have been transgenically enhanced for chitinolytic activity, and that express high levels of chitinolytic enzymes, exhibit the expected increase in resistance to fungal pathogens (Schickler et al., “Heterologous Chitinase Gene Expression to Improve Plant Defense Against Phytopathogenic Fungi,” Journal of Industrial Microbiology & Biotechnology 19:196-201 (1997)). Sela-Buurlage et al., “Only Specific Tobacco (Nicotiana tabacum) Chitinases and β-1,3-Glucanases Exhibit Antifungal Activity,” Plant Physiology 101:857-863 (1993) showed that only class I vacuolar chitinolytic enzymes and β-1-3-glucanase isoform from tobacco exhibited antifungal activity against Fusarium solani, while the class II isoform of both enzymes exhibited no antifungal activity.

The possibility that chitinolytic enzymes are involved in non-defensive roles is just beginning to be elucidated (Patil et al., “Possible Correlation Between Increased Vigour and Chitinase Activity Expression in Tobacco,” Journal of Experimental Botany 48:1943-1950 (1997)). Recently, chitinolytic enzymes have been shown to be involved in the process of flowering (Neale et al., “Chitinase, β-1,3-Glucanase, Osmotin, and Extensin are Expressed in Tobacco Explants; During Flower Formation,” Plant Cell 2:673-684 (1990)), reproduction (Leung et al., “Involvement of Plant Chitinase in Sexual Reproduction of Higher Plants,” Phytochemistrg 31:1899-1900 (1992)), germination (Vogeli-Lange et al., “Evidence for a Role of β-1,3-Glucanase in Dicot Seed Germination,” The Plant Journal 5:273-278 (1994); and Wu et al., “Molecular Analysis of Two cDNA Clones Encoding Acidic Class I Chitinase in Maize,” Plant Physiology 105:1097-1105 (1994)), somatic embryogenesis (de Jong et al., “A Carrot Somatic Embryo Mutant is Rescued by Chitinase,” The Plant Cell 4:425-433 (1992); Dong et al., “Endochitinase and β-1,3-Glucanase Genes are Developmentally Regulated During Somatic Embryogenesis in Picea glauca,” Planta 201:189-94 (1997); van Hengel et al., “Expression Pattern of the Carrot EP3 Endochitinase Genes in Suspension Culture and in Developing Seeds,” Plant Physiology 117:43-53(1998)), plant growth (Patil et al., “Possible Correlation Between Increased Vigour and Chitinase Activity Expression in Tobacco,” Journal of Experimental Botany 48:1943-1950 (1997); Spaink et al., “Rhizobial Lipo-oligosaccharide Signals and Their Role in Plant Morphogenesis: Are Analogous Lipophilic Chitin Derivatives Produced by the Plant?,” Australian Journal of Plant Physiology 20:381-392 (1993)), fruit ripening (Robinson et al., “A Class IV Chitinase is Highly Expressed in Grape Berries During Ripening,” Plant Physiology (Rockville) 114:771-778 (1997)), and senescence (Hanfrey et al., “Leaf Senescence in Brassica napus: Expression of Genes Encoding Pathogenesis-Related Proteins,” Plant Molecular Biology 30:597-609 (1996)).

Another aspect that has not been elucidated is the target substrate in plants for the plant chitinolytic enzymes. Until recently, it was believed that no substrates for chitinolytic enzymes were present in the plants. Flach et al., “What's New in Chitinase Research?,” Experientia 48:701-716 (1992), reported that the interest in plant chitinolytic enzymes is partly due to the probable absence of natural substrates in the plant itself. However, chitinolytic enzymes catalyze the hydrolysis of chitin, a linear homopolymer of β1-4-linked N-acetylglucosamine (GlcNAc) residues. Immunological studies have reveal the presence of GlcNAc residues in secondary cell wall of plants, probably in form of glycolipids (Benhamou et al., “Attempted Localisation of a Substrate for Chitinase in Plant Cell Reveals Abundant N-acetyl-D-glucosamine Residues in Secondary Wall,” Biol. Cell 67:341-350 (1989); and Benhamou et al., “Subcellular Localization of Chitinase and of its Potential Substrate in Tomato Root Tissues Infected by Fusarium oxysporum f. sp. radicis-lycopersici,” Plant Physiol. 92:1108-1120 (1990)). In addition, proteins associated with the tobacco nuclear pore complex that have oligosaccharides attached to the terminal N-acetyl glucosamine residues have been identified (Benhamou et al., “Attempted Localisation of a Substrate for Chitinase in Plant Cell Reveals Abundant N-acetyl-D-glucosamine Residues in Secondary Wall,” Biol. Cell 67:341-350(1989); and Heese-Peck et al., “Plant Nuclear Pore Complex Proteins are Modified by Novel Oligosaccharides with Terminal N-acetylglucosamine,” The Plant Cell 7:1459-147 (1995)). Those results have led to the belief that chitinolytic enzymes may have non-defensive functions such as digestion of the plant cell wall material (Vogeli-Lange et al., “Evidence for a Role of β-1,3-Glucanase in Dicot Seed Germination,” The Plant Journal 5:273-278 (1994)), cell division, differentiation and development (Collinge et al., “Plant Chitinases,” Plant J. 3:31-40 (1993); and Patil et al., “Possible Correlation Between Increased Vigour and Chitinase Activity Expression in Tobacco,” Journal of Experimental Botany 48:1943-1950 (1997)).

Thus, the role of endochitinase and chitobiosidase in plant growth and development needs to be further elucidated, and methods for the application of these enzymes for the enhancement of plant propagation are desirable.

The present invention is directed to overcoming these and other deficiencies in the art.

### SUMMARY OF THE INVENTION

The present invention relates to a method of promoting early flowering in plants by providing a transgenic plant or plant seed transformed with a DNA molecule encoding a chitinolytic enzyme having chitinolytic activity and growing the transgenic plant or transgenic plants produced from the transgenic plant seeds under conditions effective to promote early flowering in the plants.

The present invention also relates to a method of promoting yield from plants by providing a transgenic plant or plant seed transformed with a DNA molecule encoding a chitinolytic enzyme and growing the transgenic plant or transgenic plants produced from the transgenic plant seeds under conditions effective to promote yield from the plants.

The present invention also relates to a method of reducing plant size by providing a transgenic plant or plant seed transformed with a DNA molecule encoding a chitinolytic enzyme and growing the transgenic plant or transgenic plants produced from the transgenic plant seeds under conditions effective to reduce growth of the plants.

This invention also relates to a method of promoting early flowering in plants by applying a chitinolytic enzyme having chitinolytic activity to a plant or plant seed under conditions effective to promote early flowering.

The present invention also discloses a method of promoting yield from plants by applying a chitinolytic enzyme having chitinolytic activity to a plant or plant seed under conditions effective to promote yield.

In addition, this invention relates to a method of reducing plant size by applying a chitinolytic enzyme having chitinolytic activity to a plant or plant seed under conditions effective to reduce plant size.

This invention also relates to transgenic plants and seeds produced by transformation with a DNA molecule encoding a chitinolytic enzyme wherein the DNA molecule is effective to promote early flowering, reduce plant growth and/or increase yield.

### BRIEF DESCRIPTION OF THE DRAWINGS

FIG. 1 shows the endochitinolytic activity in Set I tomato plants at 30, 45, and 105 days post-planting. Black bars represent the average endochitinolytic activity in control Beefmaster. Gray bars represent the average endochitinolytic activity T2 transgenic Beefmaster B1 line. Vertical lines indicate ±1 SE. Columns associated with a different letter are significantly different.

FIG. 2 shows the chitobiosidase activity in Set I tomato plants at 30, 45, and 105 days post-planting. Black bars represent the average chitobiosidase activity in control Beefmaster. Gray bars represent the average endochitinolytic activity in T2 transgenic Beefmaster B1 line. Vertical lines indicate ±1 SE. Columns associated with a different letter are significantly different.

FIG. 3 shows the endochitinolytic activity in Set II tomato plants at 30, 45, and 75 days post-planting. Black bars represent the average endochitinolytic activity in control Beefmaster. Gray bars represent the average endochitinolytic activity T2 transgenic Beefmaster B1 line. Vertical lines indicate ±1 SE. Columns associated with a different letter are significantly different.

FIG. 4 shows the chitobiosidase activity in Set II tomato plants at 30, 45, and 75 days post-planting. Black bars represent the average chitobiosidase activity in control Beefmaster. Gray bars represent the average chitobiosidase activity in T2 transgenic Beefmaster B1 line. Vertical lines indicate ±1 SE. Columns associated with a different letter are significantly different.

FIG. 5 shows the plant height (cm) in set I and set II tomato plants at 45 days post-transplanting. Black bars represent the average plant height for control Beefmaster. Gray bars represent the average plant height for T2 transgenic Beefmaster B1 line. Vertical lines indicate ±1 SE. Columns associated with a different letter are significantly different.

FIG. 6 shows the correlation between endochitinase activity and plant height 30 days post-planting of tomato plants. Data from Set 1 and Set 2 were pooled. The top two data points correspond to the transgenic plants; the bottom two data points correspond to the control plants. Y=−0.546 log (x)+0.923, r2=0.989.

FIG. 7 shows the differences in flowering time (days) in Set I and Set II tomato plants post-transplanting. Black bars represent the average time to flowering for control Beefmaster. Gray bars represent the average time to flowering for T2 transgenic Beefmaster B1 line. Vertical lines indicate 1 SE. Columns associated with a different letter are significantly different.

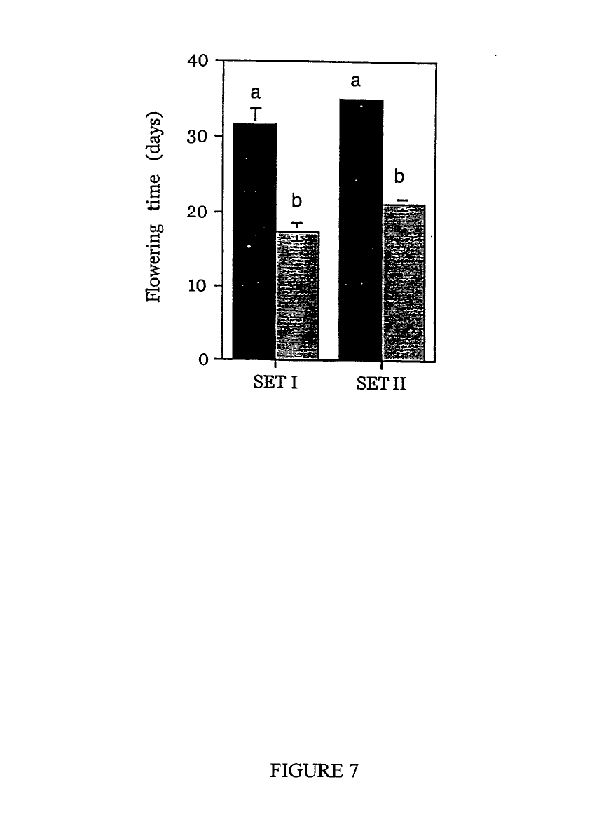

FIG. 8 shows the Southern blot of tomato using a T-DNA probe synthesized from the construct pS.a-endochitinase-chitobiosidase. Lane 1 (plasmid 15 pg) was loaded with 15 pg of pS.a-endochitinase-chitobiosidase digested with HindIII. All the other lanes were loaded with 15 μg of DNA digested with HindIII. Lane 2 (Bm control) was loaded with a sample from Beefmaster non-transgenic plant. All the other lines correspond to T1 (transgenic) lines.

FIG. 9 shows a Southern blot of tomato using a chitobiosidase probe synthesized from the construct pBS chitobiosidase. Lane 1 (plasmid 15 pg) was loaded with 15 pg of pS.a-endochitinase-chitobiosidase digested with HindIII. All the other lanes were loaded with 15 μg of DNA digested with HindIII. Lane 2 (BM control) was loaded with a sample from Beefmaster non-transgenic plant. All the other lines correspond to T1 (transgenic) lines.

FIG. 10 shows a tomato Southern blot using the endochitinase probe. Lane 1 (plasmid 15 pg) was loaded with the 15 pg of pS.a-endochitinase digested with HindIII. All the other lanes were loaded with 15 μg of DNA digested with HindIII. Lane 2 (Bm control) was loaded with a sample from Beefmaster non-transgenic plant. All the other lines correspond to T1 (transgenic) lines.

FIG. 11 shows the endochitinolytic activity in the tomato T1 transgenic lines (B1, C1, F1, F2, F3, H1 and H2) and control plants (non-transgenic). Black bars represent the average endochitinolytic activity at 30 days post-planting. Gray bars represent the average endochitinolytic activity at 45 days post-planting. Vertical lines indicate ±1 SE. Columns associated with a different letter are significantly different.

FIG. 12 shows the chitobiosidase activity in the tomato T1 transgenic lines (B1, C1, F1, F2, F3, H1 and H2) and control plants (non-transgenic). Black bars represent the average chitobiosidase activity at 30 days post-planting. Gray bars represent the average chitobiosidase activity at 45 days post-planting. Vertical lines indicate ±1 SE. Columns associated with a different letter are significantly different.

FIG. 13 shows the differences in plant height (cm) in transgenic tomato T1 lines (B1, C1, F1, F2, F3, H1 and H2) and control plants 45 days post-transplanting. The bars represent the average plant height for each T1 transgenic line and control plants. Vertical lines indicate ±1 SE. Columns associated with a different letter are significantly different.

FIG. 14 shows the correlation between endochitinase activity and plant size (cm) at 45 post-planting in different tomato T1 plants and control plants. Y=−1,443 log (x)+2.423 r2=0.785.

FIG. 15 shows the differences in flowering time (days) in transgenic tomato T1 lines (B1, C1, F1, F2, F3, H1 and H2) and control plants post-transplanting. The Bars represent the average plant height for each T1 transgenic line and control plants. Vertical lines indicate ±1 SE. Columns associated with a different letter are significantly different.

FIG. 16 shows the correlation between endochitinase activity and flowering time (days) 45 days post-planting in different tomato T1 plants and control (non-transgenic plants. Y=−8.443 log(x)+15.8 r2=0.832

FIG. 17 shows the effect of trimming on flowering time (days) in control plants and T2 transgenic B1-1 line. Black bars represent the average flowering time in plants that were trimmed. Gray bars represent the average flowering time in plants that were not trimmed. Vertical lines indicate ±1 SE. Columns associated with a different letter are significantly different.

FIG. 18 shows the effect of trimming on the number of fruits produced 60 days post-transplanting of control plants and T2 transgenic B1-1 tomato line plants. Black bars represent the mean number of fruits on plants that were trimmed. The gray bars represent the mean number of fruits on non-trimmed plants. Vertical lines indicate ±1 SE. Columns associated with the same letter are significantly different.

### DETAILED DESCRIPTION OF THE INVENTION

The present invention relates to a method of promoting early flowering in plants by providing a transgenic plant or plant seed transformed with a DNA molecule encoding a chitinolytic enzyme having chitinolytic activity and growing the transgenic plant or transgenic plants produced from the transgenic plant seeds under conditions effective to promote early flowering in the plants.

An example of a chitinolytic enzyme suitable for use in the present invention is a chitobiosidase having an amino acid sequence of SEQ. ID. No. 1 as follows:

```
Ala Pro Ala Ala Val Pro Ala His Ala Val Thr Gly Tyr Trp Gln Asn
    1 5 10 15
Phe Asn Asn Gly Ala Thr Val Gln Thr Leu Ala Asp Val Pro Asp Ala
                          20 25 30
Tyr Asp Ile Ile Ala Val Ser Phe Ala Asp Ala Thr Ala Asn Ala Gly
                  35 40 45
Glu Ile Thr Phe Thr Leu Asp Ser Val Gly Leu Gly Gly Tyr Thr Asp
          50 55 60
Glu Gln Phe Arg Ala Asp Leu Ala Ala Lys Gln Ala Asp Gly Lys Ser
65 70 75 80
Val Ile Ile Ser Val Gly Gly Glu Lys Gly Ala Val Ala Val Asn Asp
                                  85 90 95
Ser Ala Ser Ala Gln Arg Phe Ala Asp Ser Thr Tyr Ala Leu Met Glu
                        100 105 110
Glu Tyr Gly Phe Asp Gly Val Asp Ile Asp Leu Glu Asn Gly Leu Asn
                115 120 125
Ser Thr Tyr Met Thr Glu Ala Leu Thr Lys Leu His Glu Lys Ala Gly
        130 135 140
Asp Gly Leu Val Leu Thr Met Ala Pro Gln Thr Ile Asp Met Gln Ser
145 150 155 160
Pro Glu Asn Glu Tyr Phe Lys Thr Ala Leu Val Thr Lys Asp Phe Leu
                                165 170 175
Thr Ala Val Asn Met Gln Tyr Tyr Asn Ser Gly Ser Met Leu Gly Cys
                        180 185 190
Asp Gly Gln Val Tyr Ala Gln Gly Thr Val Asp Phe Leu Thr Ala Leu
                195 200 205
Ala Cys Ile Gln Leu Glu Asn Gly Leu Asp Ala Ser Gln Val Gly Ile
        210 215 220
Gly Val Pro Ala Ser Pro Lys Ala Ala Gly Gly Gly Tyr Val Glu Pro
225 230 235 240
Ser Val Val Asn Asp Ala Leu Asp Cys Leu Thr Arg Gly Thr Gly Cys
                        245 250 255
Gly Ser Phe Lys Pro Glu Lys Thr Tyr Pro Ala Leu Arg Gly Ala Met
                        260 265 270
Thr Trp Ser Thr Asn Trp Asp Ala Asp Thr Gly Asn Ala Trp Ser Asn
                275 280 285
Val Val Gly Pro His Val Asp Asp Leu Pro
        290 295
```

This amino acid encodes a chitobiosidase isolated from Streptomyces albidoflavus which has a molecular mass of 34 kD and an isoelectric point of less than 3.0.

The chitobiosidase isolated from Streptomyces albidoflavus having an amino acid sequence of SEQ. ID. No. 1 is encoded by a nucleic acid molecule having a nucleotide sequence of SEQ. ID. No. 2 as follows:

| | |
|---|---|
| gcggccgctc cgggcggacg accgtacgga ctcctcggcc gacccctgcg ggaacccttg | 60 |
| acaaccccat tggtctggac cagtttggtg cccatcgcgg tggccaccgt gcgccaactc | 120 |
| cccgccccct cccgggtgga gggccccgtc ggcgcgtccc cccacgtccg tgactccccc | 180 |
| caccggaggc agcagtggta cgcacctacc cccttccgca ccccggccgg cgcccctcca | 240 |
| cgcccggcct ccaccgcagg ggccggctga ccgccgccct caccgcggcc gtcctcggcg | 300 |
| cctccgggct cgccctcacc ggccccgcga ccgccggcga gggggccccc gccgcccagg | 360 |
| ccgccccgga cgccgtaccg gcccacgcgg tgaccggtta ctggcagaac ttcaacaacg | 420 |
| gcgcgaccgt gcagaccctc gccyacgtgc cggacgccta cgacatcatc gccgtctcct | 480 |
| tcgccgacgc cacggccaac gcgggcgaga tcaccttcac cctcgactcg gtcgggctcg | 340 |
| gcggctacac cgacgagcag ttccgcgccg acctcgccgc caagcaggcc gacggcaagt | 600 |
| cggtgatcat ctcggtcggc ggcgagaagg gcgcggtcgc cgtcaacgac agcgcctccg | 660 |
| cccagcgctt cgccgacagc acctacgcgc tgatggagga gtacggcttc gacggcgtcg | 720 |
| acatcgacct ggagaacggc ctcaactcca cctacatgac cgaggccctc accaagctcc | 780 |
| acgagaaggc cggggacggc ctggtcctca ccatggcgcc gcagaccatc gacatgcagt | 840 |
| cgcccgagaa cgagtacttc aagacggcgc tggtcacgaa agacttcctg accgccgtca | 900 |
| acatgcagta ctacaacagc ggctcgatgc tcggctgcga cggccaggtc tacgcgcagg | 960 |
| gcaccgtcga cttcctcacc gcgctcgcct gcatccagct ggagaacggt ctcgacgcct | 1020 |
| cccaggtcgg catcggtgtc cccgcctccc cgaaggcggc cggcggcggc tacgtcgagc | 1080 |
| cctccgtggt caacgacgcg ctggactgcc tgacccgggg caccggttgt ggctcgttca | 1140 |
| agccggagaa gacctacccg gcgctgcgtg gcgccatgac ctggtcgacc aactgggacg | 1200 |
| ccgacaccgg caacgcctgg tcgaacgtgg tcggcccgca cgtcgacgac ctgccgtaac | 1260 |
| cccggagccg ggcacccgtc cgcttccccc gcac | 1294 |

An example of an endochitinase suitable for use in the present invention has an amino acid sequence of SEQ. ID. No. 3 as follows:

```
Gly Pro Gly Pro Gly Pro Arg Glu Lys Ile Asn Leu Gly Tyr Phe Thr
    1 5 10 15
Glu Trp Gly Val Tyr Gly Arg Asn Tyr His Val Lys Asn Leu Val Thr
                          20 25 30
Ser Gly Ser Ala Glu Lys Ile Thr His Ile Asn Tyr Ser Phe Gly Asn
                  35 40 45
Val Gln Gly Gly Lys Cys Thr Ile Gly Asp Ser Phe Ala Ala Tyr Asp
          50 55 60
Lys Ala Tyr Thr Ala Ala Glu Ser Val Asp Gly Val Ala Asp Thr Trp
  65 70 75 80
Asp Gln Pro Leu Arg Gly Asn Phe Asn Gln Leu Arg Lys Leu Lys Ala
                                  85 90 95
Lys Tyr Pro His Ile Lys Val Leu Trp Ser Phe Gly Gly Trp Thr Trp
                        100 105 110
Ser Gly Gly Phe Thr Asp Ala Val Lys Asn Pro Ala Ala Phe Ala Lys
                115 120 125
Ser Cys His Asp Leu Val Glu Asp Pro Arg Trp Ala Asp Val Phe Asp
        130 135 140
Gly Ile Asp Leu Asp Trp Glu Tyr Pro Asn Ala Cys Gly Leu Ser Cys
145 150 155 160
Asp Ser Ser Gly Pro Ala Ala Leu Lys Asn Met Val Gln Ala Met Arg
                                165 170 175
Ala Gln Phe Gly Thr Asp Leu Val Thr Ala Ala Ile Thr Ala Asp Ala
                        180 185 190
Ser Ser Gly Gly Lys Leu Asp Ala Ala Asp Tyr Ala Gly Ala Ala Gln
                195 200 205
Tyr Phe Asp Trp Tyr Asn Val Met Thr Tyr Asp Phe Phe Gly Ala Trp
        210 215 220
Asp Lys Thr Gly Pro Thr Ala Pro His Ser Ala Leu Asn Ser Tyr Ser
225 230 235 240
Gly Ile Pro Lys Ala Asp Phe His Ser Ala Ala Ala Ile Ala Lys Leu
                                245 250 255
Lys Ala Lys Gly Val Pro Ala Ser Lys Leu Leu Leu Gly Ile Gly Phe
                        260 265 270
Tyr Gly Arg Gly Trp Thr Gly Val Thr Gln Asp Ala Pro Gly Gly Thr
                275 280 285
Ala Thr Gly Pro Ala Thr Gly Thr Tyr Glu Ala Gly Ile Glu Asp Tyr
        290 295 300
Lys Val Leu Lys Asn Thr Cys Pro Ala Thr Gly Thr Val Gly Gly Thr
305 310 315 320
Ala Tyr Ala Lys Cys Gly Ser Asn Trp Trp Ser Tyr Asp Thr Pro Ala
                                325 330 335
Thr Ile Lys Thr Lys Met Thr Trp Ala Lys Asp Gln Gly Leu Gly Gly
                        340 345 350
Ala Phe Phe Trp Glu Phe Ser Gly Asp Thr Ala Gly Gly Glu Leu Val
                355 360 365
Ser Ala Met Asp Ser Gly Leu Arg
        370 375
```

This endochitinase is isolated from Streptomyces albidoflavus and has a molecular mass of 45 kD and an isoelectric point of about 6.5.

The endochitinase isolated from Streptomyces albidoflavus having an amino acid sequence of SEQ. ID. No. 3 is encoded by a nucleic acid molecule having a nucleotide sequence of SEQ. ID. No. 4 as follows:

| | |
|---|---|
| gtcgactggt acaacgtgat gacctacgac tacttcggca cctgggccgc ccagggcccg | 60 |
| acggcgcccc actcgccgct caccgcctac ccgggcatcc agggcgagca caacacctcc | 120 |
| tcggccacca tcgccaagct gcggggcaag ggcatcccgg cgaagaagct gctgctgggc | 180 |
| atcggcgcct acggccgcgg ctggaccggc gtcacccagg acgcccccgg cggcaccgcc | 240 |
| accggcccgg ccgccggcac ctacgaggcg ggcaacgagg agtaccgggt gctggccgag | 300 |
| aagtgcccgg ccaccggcac cgccggcggc accgcgtacg ccaagtgcgg cgacgactgg | 360 |
| tggagttacg acacacctga gacggtgacg ggcaagatgg cctgggcgaa gaagcagaag | 420 |
| ctcggcggtg ccttcctctg ggagttcgcc ggcgacggcg ccaagggcga tctgttcagg | 480 |
| gcgatgcacg aggggctgcg ctgaccggcc gggcactcac ccggaactga cccttcccgc | 540 |
| acggccgtcc gccgtggcac cggagctccg gtcgccgcgg cgggcggccg tgtccgcatg | 600 |
| tcgccacccc cgcgcaccag gcgcgatccg gccgaacttt cctttggtcc agacctcttg | 660 |
| acctctggtc cagacctttt ctactctcgc cccactgcgg tgggcacatc ggtcgtcggt | 720 |
| gctcacgggc gtcgcagggt tccgccccca tacgtccgga cctcttgagg agtacgcctt | 780 |
| gagtacggtt tcccccagca ccgacggcgc ccgcagccgt cccagacccc tcagccgctt | 840 |
| ccgccggcgc gcgctggccg cgctcgtcgg cctcgcggtc cccttcgccg ggatggtcgg | 900 |
| cctcgccgcc cccacccagg ccgccgaggc cgcggccgac cccagcgcct cctacaccag | 960 |
| gacgcaggac tggggcagcg gcttcgaggg caagtggacg gtgaagaaca ccggcaccgc | 1020 |
| ccccctcagc ggctggaccc tggagtggga cttccccgcc ggaaccaagg tgacctcggc | 1080 |
| ctgggacgcc gacgtcacca acaacggcga ccactggacc gcaaagaaca agagctgggc | 1140 |
| ggggagcctc gcccccggcg cctcggtcag cttcggcttc aacggcaccg gccccggcac | 1200 |
| cccctcgggc tgcaagctca acggcgcctc ctgcgacggc ggcagcgtcc ccggcgacac | 1260 |
| cccgcccacc gcccccggca cccccaccgc cagtgacctc accaagaact cggtgaagct | 1320 |
| ctcctggaag gcggccaccg acgacaaggg cgtcaagaac tacgacgtcc tgcgcgacgg | 1380 |
| cgccaaggtc gccaccgtca ccgccaccac cttcaccgac cagaacctcg cccccggcac | 1440 |
| cgactactcc tactcggtcc aggcccgcga caccgccgac cagaccggcc cggtcagcgc | 1500 |
| ccccgtcaag gtcaccaccc ccggcgacgg cacgggcccc ggccccggcc cccgcgagaa | 1560 |
| gatcaacctc ggctacttca ccgagtgggg cgtctacggc cgcaactacc acgtcaaaaa | 1620 |
| cctggtgacc tccggctccg ccgagaagat cacccacatc aactactcct tcggcaacgt | 1680 |
| ccagggcggc aagtgcacca tcggtgacag cttcgccgcc tacgacaagg cgtacaccgc | 1740 |
| cgccgagtcg gtcgacggcg tcgccgacac ctgggaccag ccgctgcgcg gcaacttcaa | 1800 |
| ccagctccgc aagctcaagg ccaagtaccc gcacatcaag gtcctctggt ccttcggcgg | 1860 |
| ctggacctgg tccggcggct tcaccgacgc cgtgaagaac ccggccgcct tcgccaagtc | 1920 |
| ctgacacgac ctggtcgagg acccgcgctg ggccgacgtc ttcgacggca tcgacctcga | 1980 |
| ctgggagtac ccgaacgcct gcggcctcag ctgcgacagc tccggtccgg ccgcgctgaa | 2040 |
| gaacatggtc caggcgatgc gcgcccagtt cggcaccgac ctggtcaccg ccgccatcac | 2100 |
| cgccgacgcc agctccggcg gcaagctcga cgccgccgac tacgcgggGg ccgcccagta | 2160 |
| cttcgactgg tacaacgtga tgacgtacga cttcttcggc gcctgggaca agaccggccc | 2220 |
| gaccgcgccc cactcggccc tgaactccta cagcggcatc cccaaggccg acttccactc | 2280 |
| ggccgccgcc atcgccaagc tcaaggcgaa gggcgtcccg gcgagcaagc tcctgctcgg | 2340 |
| catcggcttc tacggccgcg gctggaccgg cgtcacccag gacgccccgg gcggcaccgc | 2400 |
| caccggcccg gccaccggca cctacgaggc gggcatcgag gactacaagg tcctcaagaa | 2460 |
| cacctgcccc gccaccggca ccgtcggcgg caccgcgtac gccaagtgcg gcagcaactg | 2520 |
| gtggagctac gacaccccgg ccaccatcaa gaccaagatg acctgggcca aggaccaggg | 2580 |
| cctcggcggc gccttcttct gggagttcag cggtgacacc gcgggcggcg aactggtctc | 2640 |
| cgcgatggac tccggcctcc gctagccccg gaccggcacc ccgcccgaac cactagcacg | 2712 |
| acctcccccg ga | |

Fragments of the above chitinolytic enzymes are also useful for use in the present invention. Suitable fragments can be produced by several means. In one method, subclones of the gene encoding the chitinolytic enzymes of the present invention are produced by conventional molecular genetic manipulation by subcloning gene fragments. The subclones then are expressed in vitro or in vivo in bacterial cells to yield a smaller protein or peptide that can be tested for chitinolytic activity according to the procedure described below.

As an alternative, fragments of a chitinolytic enzyme can be produced by digestion of a full-length chitinolytic enzyme with proteolytic enzymes like chymotrypsin or Staphylococcus proteinase A, or trypsin. Different proteolytic enzymes are likely to cleave chitinolytic enzymes at different sites based on the amino acid sequence of the chitinolytic enzyme. Some of the fragments that result from proteolysis may be active chitinolytic enzymes.

In another approach, based on knowledge of the primary structure of the protein, fragments of a chitinolytic enzyme encoding gene may be synthesized by using the PCR technique together with specific sets of primers chosen to represent particular portions of the protein. These then would be cloned into an appropriate vector for increased expression of a truncated peptide or protein.

Chemical synthesis can also be used to make suitable fragments. Such a synthesis is carried out using known amino acid sequences for a chitinolytic enzyme being produced. Alternatively, subjecting a full length chitinolytic enzyme to high temperatures and pressures will produce fragments. These fragments can then be separated by conventional procedures (e.g., chromatography, SDS-PAGE).

Variants may also (or alternatively) be modified by, for example, the deletion or addition of amino acids that have minimal influence on the properties, secondary structure, and hydropathic nature of an enzyme. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

An example of a nucleic acid which is a suitable alternative to the nucleic acid corresponding to SEQ. ID. No. 2, and which is useful in the present invention, is the cDNA molecule which corresponds to SEQ. ID. No. 5, as follows:

| | |
|---|---|
| atggccgccg taccggccca cycggtgacc ggttactggc agaacttcaa caacggcgcg | 60 |
| accgtycaga ccctcgccga cgtgccggac gcctacgaca tcatcgccgt ctccttcgcc | 120 |
| gacgccacgg ccaacgcggg cgagatcacc ttcaccctcg actcggtcgg gctcggcggc | 180 |
| tacaccgacg agcagttccg cgccgacctc gccgccaagc aggccgacgg caagtcggtg | 240 |
| atcatctcgg tcggcggcga gaagggcgcg gtcgccgtca acgacagcgc ctccgcccag | 300 |
| cgcttcgccg acagcaccta cgcgctgatg gaggagtacg gcttcgacgg cgtcgacatc | 360 |
| gacctggaga acggcctcaa cgccacctac atgaccgagg ccctcaccaa gctccacgag | 420 |
| aaggccgggg acggcctggt cctcaccatg gcgccgcaga ccatcgacat gcagtcgccc | 480 |
| gagaacgagt acttcaagac ggcgctggtc acgaaagact tcctgaccgc cgtcaacatg | 540 |
| cagtactaca acagcggctc gatgctcggc tgcgacggcc aggtctacgc gcagggcacc | 600 |
| gtcgacttcc tcaccgcgct cgcctgcatc cagctggaga acggtctcga cgcctcccag | 660 |
| gtcggcatcg gtgtccccgc ctccccgaag gcggccggcg gcggctacgt cgagccctcc | 720 |
| gtggtcaacg acgcgctgga ctgcctgacc cggggcaccg gttgtggctc gttcaagccg | 780 |
| gagaagacct acccggcgct gcgtggcgcc atgacctggt cgaccaactg ggacgccgac | 840 |
| accggcaacg cctggtcgaa cgtggtcggc ccgcacgtcg acgacctgcc gtaaccccgg | 900 |
| agccg | 905 |

This nucleic acid, isolated from Streptomyces albidoflavus, encodes a chitobiosidase enzyme.

An example of a nucleic acid which is a suitable alternative to the nucleic acid corresponding to SEQ. ID. No. 4, and which is useful in the present invention, is the cDNA molecule which corresponds to SEQ. ID. No. 6, as follows:

| | |
|---|---|
| atgggctact tcaccgagtg gggcgtctac ggccgcaact accacgtcaa aaacctggtg | 60 |
| acctccggct ccgccgagaa gatcacacac atcaactact ccttcggcaa cgtccagggc | 120 |
| ggcaagtgca ccatcggtga cagcttcgcc gcctacgaca aggcgtacac cgccgccgag | 180 |
| tcggtcgacg gcgtcgccga cacctgggac cagccgctgc gcggcaactt caaccagctc | 240 |
| cgcaagctca aggccaagta cccgcacatc aaggtcctct ggtccttcgg cggctggacc | 300 |
| tggtccggcg gcttcaccga cgccgtgaag aacccggccg ccttcgccaa gtcctgccac | 360 |
| gacctggtcg aggacccgcg ctgggccgac gtcttcgacg gcatcgacct cgactgggag | 420 |
| tacccgaacg cctgcggcct cagctgcgac agctccggtc cggccgcgct gaagaacatg | 480 |
| gtccaggcga tgcgcgccca gttcggcacc gacctggtca ccgccgccat caccgccgac | 540 |
| gccagctccg gcggcaagct cgacgccgcc gactacgcgg gcgccgccca gtacttcgac | 600 |
| tggtacaacg tgatgacgta cgacttcttc ggcgcctggg acaagaccgg cccgaccgcg | 660 |
| ccccactcgg ccctgaactc ctacagcggc atccccaagg ccgacttcca ctcggccgcc | 720 |
| gccatcgcca agctcaaggc gaagggcgtc ccggcgagca agctcctgct cggcatcggc | 780 |
| ttctacggcc gcggctggac cggcgtcacc caggacgccc cgggcggcac cgccaccggc | 840 |
| ccggccaccg gcacctacga ggcgggcatc gaggactaca aggtcctcaa gaacacctgc | 900 |
| cccgccaccg gcaccgtcgg cggcaccgcg tacgccaagt gcggcagcaa ctggtggagc | 960 |
| tacgacaccc cggccaccat caagaccaag atgacctggg ccaaggacca gggcctcggc | 1020 |
| ggcgccttct tctgggagtt cagcggtgac accgcgggcg gcgaactggt ctccgcgatg | 1080 |
| gactccggcc tccgctagtc tagacgg | 1107 |

This nucleic acid, isolated from Streptomyces albidoflavus, encodes an endochitinase enzyme.

Another example of a nucleic acid which is a suitable alternative to SEQ. ID. No. 2, and which is useful in the present invention, is the cDNA molecule which corresponds to SEQ. ID. No. 7, as follows:

| | |
|---|---|
| atggccgccg taccggccca cgcggtgacc ggttactggc agaacttcaa caacggcgcg | 60 |
| accgtgcaga ccctcgccga cgtgccggac gcctacgaca tcatcgccgt ctccttcgcc | 120 |
| gacgccacgg ccaacgcggg cgagatcacc ttcaccctcg actcggccgg gctcggcggc | 180 |
| tacaccgacg agcagttccg cgccgacctc gccgccaagc aggccgacgg caagtcggtg | 240 |
| atcatctcgg tcggcggcga gaagggcgcg gtcgccgtca acgacagcgc ctccgcccag | 300 |
| cgcttcgccg acagcaccta cgcgctgatg gaggagtacg gcttcgacgg cgtcgacatc | 360 |
| gacctggaga acggcctcaa ctccacctac atgaccgagg ccctcaccaa gctccacgag | 420 |
| aaggccgggg acgggctggt cctcaccatg gcgccgcaga ccatcgacat gcagtcgccc | 480 |
| gagaacgagt acttcaagac ggcgctggcc acyaaagact ttctgaccgt cgtcaacatg | 540 |
| cagtactaca acagcggttc gatgctcggc tgcaacggcc aggtctacgc gcagggcacc | 600 |
| gtcgaattcc tcaccgcgct cgcctgcatc caactggaga acggtctcga cgcctcccag | 660 |
| gtcggcatcg gcgtccccgc ctccccgaag gcgggcggcg gcggctactt cgagccctcc | 720 |
| gtggtcaacg acccctggac tgccttgacc cggggcaccg gttgtggctc gttcaagccg | 780 |
| gagaagacct acccggcgct gcgtggcgcc atgacctggt cgaccaactg ggacgccgac | 840 |
| accggcaacg cctggtcgaa cgtggtcggc ccgcacgtcg acgacctgcc gtaatctaga | 900 |
| cggat | 905 |

This nucleic acid, isolated from Streptomyces albidoflavus, encodes a chitobiosidase enzyme.

Another example of a nucleic acid which is a suitable alternative to SEQ. ID. No. 4, and which is useful in the present invention, is the cDNA molecule which corresponds to SEQ. ID. No. 8, as follows:

| | |
|---|---|
| atgggctact tcaccgagtg gggcgtctac ggccgcaact accacgtcaa aagcctggtg | 60 |
| acctccggct ccgccgagaa gatcacccac atcaactact ccttcggcaa cgtccagggc | 120 |
| ggcaagtgca ccatcggtga cagcttcgcc gcctacgaca aggcgtacac cgccgccgag | 180 |
| tcggtcgacg gcgtcgccga cacctgggac cagccgctgc gcggcaactt caaccagctc | 240 |
| cgcaagctca aggccaagta cccgcacatc aaggtcctct ggtccttcgg cggctggact | 300 |
| ggtccggcgg cttcaccgag ccgtgaagaa cccggccgcc ttcgccaagt cctgccacga | 360 |
| cctggtcgag cgacccgcgc tgggccgacg tcttcgacgg catcgacctc gactgggagt | 420 |
| acccgaacgc ctgcggcctc agctgcgaca gctccggtcc ggccgcgctg aagaacatgg | 480 |
| tccaggcgat gcgcgcccag ttcggcaccg acctggtcac cgccgccatc accgccgacg | 540 |
| ccagctccgg cggcaagctc gacgccgccg actacgcggg cgccgcccag tacttcgact | 600 |
| ggtacaacgt gatgacgtac gacttcttcc gcgcctggga caagaccggc ccgaccgcgc | 660 |
| cccactcggc cctgaaattc tacagcggca tccccaaggc cgaattccaa tcggccgcgc | 720 |
| catcgccaag ctcaaggcga agggcgtccc ggcgagcaag ctcctgctcg gcatcggctt | 780 |
| ctacggccgc ggctggaccg gcgtcaccca ggacgccccg ggcggcaccg ccaccggccc | 840 |
| ggccaccggc acctacgagg cgggcatcga ggactacaag gtcctcaaga acacctgccc | 900 |
| cgccaccggc accgtcggcg gcaccgcgta cgccaagtgc ggcagcaact ggtggagcta | 960 |
| cgacaccccg gccaccatca agaccaagat gacctgggcc aaggaccagg gcctcggcgg | 1020 |
| cgccttcttc tgggagttca gcggtgacac cgcgggcggc gaactggtct ccgcgatgac | 1080 |
| tccggcctcc gctagtctag acgg | 1104 |

This nucleic acid, isolated from Streptomyces albidoflavus, encodes an endochitinase enzyme.

Suitable DNA molecules are those that hybridize to a DNA molecule comprising a nucleotide sequence of SEQ. ID. Nos. 2, 4, 5, 6, 7, and 8 under stringent conditions. An example of suitable stringency conditions is when hybridization is carried out at a temperature of 65° C. for 20 hours in a buffer containing 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA. Less stringent hybridization conditions may be carried out using the hybridization buffer just above, at a temperature of 56° C.

The nucleic acid molecule encoding one or more of the chitinolytic enzymes of the present invention can be incorporated in cells using conventional recombinant DNA technology. Generally, this involves inserting the nucleic acid molecule into an expression system to which the nucleic acid molecule is heterologous (i.e., not normally present). The heterologous nucleic acid molecule is inserted into the expression system or vector in proper sense orientation and correct reading frame. The vector contains the necessary elements for the transcription and translation of the inserted protein-coding sequences.

The nucleic acid molecules of the present invention may be inserted into any of the many available expression vectors and cell systems using reagents that are well known in the art. Suitable vectors include, but are not limited to, the following viral vectors such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Second Edition, Cold Spring Harbor Press, NY (1989), and Ausubel, F. M. et al. (1989) Current Protocols in Molecular Biology, John Wiley & Sons, New York, N.Y., which are hereby incorporated by reference.

In preparing a DNA vector for expression, the various DNA sequences may normally be inserted or substituted into a bacterial plasmid. Any convenient plasmid may be employed, which will be characterized by having a bacterial replication system, a marker which allows for selection in a bacterium and generally one or more unique, conveniently located restriction sites. Numerous plasmids, referred to as transformation vectors, are available for plant transformation. The selection of a vector will depend on the preferred transformation technique and target species for transformation. A variety of vectors are available for stable transformation using Agrobacterium tumefaciens, a soilborne bacterium that causes crown gall. Crown gall are characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTI, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of Agrobacterium tumefaciens. Fraley, et al., “Expression of Bacterial Genes in Plant Cells,” Proc. Nat'l Acad. Sci., 80:4803-4807 (1983), which is hereby incorporated by reference.

Further improvement of this technique led to the development of the binary vector system. Bevan, M., “Binary Agrobacterium Vectors for Plant Transformation,” Nucleic Acids Res. 12:8711-8721 (1984), which is hereby incorporated by reference. In this system, all the T-DNA sequences (including the borders) are removed from the pTi, and a second vector containing T-DNA is introduced into Agrobacterium tumefaciens. This second vector has the advantage of being replicable in E. coli as well as A. tumefaciens, and contains a multiclonal site that facilitates the cloning of a transgene. An example of a commonly used vector is pBin19. Frisch, et al., “Complete Sequence of the Binary Vector Bin19,” Plant Molec. Biol. 27:405-409 (1995), which is hereby incorporated by reference. Any appropriate vectors now known or later described for genetic transformation are suitable for use with the present invention.

U.S. Pat. No. 4,237,224 issued to Cohen and Boyer, which is hereby incorporated by reference, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture.

In one aspect of the present invention, the nucleic acid molecule of the present invention is incorporated into an appropriate vector in the sense direction, such that the open reading frame is properly oriented for the expression of the encoded protein under control of a promoter of choice. Single or multiple nucleic acids of the present invention may be ligated into an appropriate vector.

Certain “control elements” or “regulatory sequences” are also incorporated into the vector-construct. Those non-translated regions of the vector, promoters, 5′ and 3′ untranslated regions-which interact with host cellular proteins to carry out transcription and translation. Such elements may vary in their strength and specificity. Depending on the vector system and host utilized, any number of suitable transcription and translation elements, including constitutive and inducible promoters, may be used.

A constitutive promoter is a promoter that directs expression of a gene throughout the development and life of an organism. Examples of some constitutive promoters that are widely used for inducing expression of transgenes include the nopoline synthase (NOS) gene promoter, from Agrobacterium tumefaciens, (U.S. Pat. No. 5,034,322 issued to Rogers et al., which is hereby incorporated by reference), the cauliflower mosaic virus (CaMv) 35S and 19S promoters (U.S. Pat. No. 5,352,605 issued to Fraley et al., which is hereby incorporated by reference), those derived from any of the several actin genes, which are known to be expressed in most cells types (U.S. Pat. No. 6,002,068 issued to Privalle et al., which is hereby incorporated by reference), and the ubiquitin promoter, which is a gene product known to accumulate in many cell types.

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, growth regulator, herbicide or phenolic compound, or a physiological stress directly imposed upon the plant such as cold, heat, salt, toxins, or through the action of a pathogen or disease agent such as a virus or fungus. A plant cell containing an inducible promoter may be exposed to an inducer by externally applying the inducer to the cell or plant such as by spraying, watering, heating, or by exposure to the operative pathogen. An example of an appropriate inducible promoter for use in the present invention is a glucocorticoid-inducible promoter (Schena et al., “A Steroid-Inducible Gene Expression System for Plant Cells,” Proc. Natl. Acad. Sci. 88:10421-5 (1991), which is hereby incorporated by reference). Expression of the transgene-encoded protein is induced in the transformed plants when the transgenic plants are brought into contact with nanomolar concentrations of a glucocorticoid, or by contact with dexamethasone, a glucocorticoid analog. Schena et al., “A Steroid-Inducible Gene Expression System for Plant Cells,” Proc. Natl. Acad. Sci. USA 88:10421-5 (1991); Aoyama et al., “A Glucocorticoid-Mediated Transcriptional Induction System in Transgenic Plants,” Plant J. 11: 605-612 (1997), and McNellis et al., “Glucocorticoid-Inducible Expression of a Bacterial Avirulence Gene in Transgenic Arabidopsis Induces Hypersensitive Cell Death, Plant J. 14(2):247-57 (1998), which are hereby incorporated by reference. In addition, inducible promoters include promoters that function in a tissue specific manner to regulate the gene of interest within selected tissues of the plant. Examples of such tissue specific promoters include seed, flower, or root specific promoters as are well known in the field (U.S. Pat. No. 5,750,385 issued to Shewmaker et al., which is hereby incorporated by reference).

The DNA construct of the present invention also includes an operable 3′ regulatory region, selected from among those which are capable of providing correct transcription termination and polyadenylation of mRNA for expression in the host cell of choice, operably linked to a DNA molecule which encodes for a protein of choice. A number of 3′ regulatory regions are known to be operable in plants. Exemplary 3′ regulatory regions include, without limitation, the nopaline synthase (“nos”) 3′ regulatory region (Fraley, et al., “Expression of Bacterial Genes in Plant Cells,” Proc. Nat'l Acad. Sci. USA 80:4803-4807 (1983), which is hereby incorporated by reference) and the cauliflower mosaic virus (“CaMV”) 3′ regulatory region (Odell, et al., “Identification of DNA Sequences Required for Activity of the Cauliflower Mosaic Virus 35S Promoter,” Nature 313(6005):810-812 (1985), which is hereby incorporated by reference). Virtually any 3′ regulatory region known to be operable in plants would suffice for proper expression of the coding sequence of the nucleic acid of the present invention.

The vector of choice, promoter, and an appropriate 3′ regulatory region can be ligated together to produce the plasmid of the present invention using well known molecular cloning techniques as described in Sambrook et al., Molecular Cloning: A Laboratory Manual, Second Edition, Cold Spring Harbor Press, NY (1989), and Ausubel, F. M. et al. (1989) Current Protocols in Molecular Biology, John Wiley & Sons, New York, N.Y., which are hereby incorporated by reference.

Once the DNA construct of the present invention has been prepared, it is ready to be incorporated into a host cell. Recombinant molecules can be introduced into cells via transformation, particularly transduction, conjugation, mobilization, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference. Suitable host cells include, but are not limited to, bacteria, virus, yeast, mammalian cells, insect, plant, and the like. Preferably the host cells are either a bacterial cell or a plant cell.

One approach to transforming plant cells with a DNA construct of the present invention is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all issued to Sanford, et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells. Other variations of particle bombardment, now known or hereafter developed, can also be used.

Transient expression in protoplasts allows quantitative studies of gene expression since the population of cells is very high (on the order of 10°). To deliver DNA inside protoplasts, several methodologies have been proposed, but the most common are electroporation (Fromm et al., “Expression of Genes Transferred Into Monocot and Dicot Plants by Electroporation,” Proc. Natl. Acad. Sci. USA 82:5824-5828 (1985), which is hereby incorporated by reference) and polyethylene glycol (PEG) mediated DNA uptake (Krens et al., “In Vitro Transformation of Plant Protoplasts with Ti-Plasmid DNA,” Nature 296:72-74 (1982), which is hereby incorporated by reference). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the gene construct of the present invention into a host cell is fusion of protoplasts with other entities, either minicells, cells, lysosomes, or other fusible lipid-surfaced bodies that contain the chimeric gene (Fraley, et al., “Entrapment of a Bacterial Plasmid in Phospholipid Vesicles: Potential for Gene Transfer,” Proc. Natl. Acad. Sci. USA, 76:3348-52 (1979), which is hereby incorporated by reference).

Stable transformants are preferable for the methods of the present invention. An appropriate method of stably introducing the DNA construct into plant cells is to infect a plant cell with Agrobacterium tumefaciens or Agrobacterium rhizogenes previously transformed with the DNA construct. Under appropriate conditions known in the art, the transformed plant cells are grown to form shoots or roots, and develop further into plants.

Plant tissues suitable for transformation include, but are not limited to, floral buds, leaf tissue, root tissue, meristems, zygotic and somatic embryos, megaspores, and anthers.

After transformation, the transformed plant cells can be selected and regenerated. Preferably, transformed cells are first identified using a selection marker simultaneously introduced into the host cells along with the DNA construct of the present invention. A widely used reporter gene for gene fusion experiments has been uidA, a gene from E. coli that encodes the β-glucuronidase protein, also known as GUS (Jefferson et al., “GUS Fusions: βGlucuronidase as a Sensitive and Versatile Gene Fusion Marker in Higher Plants,” EMBO J. 6:3901-3907 (1987), which is hereby incorporated by reference). In order to evaluate GUS activity, several substrates are available. The most commonly used are 5 bromo-4 chloro-3 indolyl glucuronide (X-Glue) and 4 methyl-umbelliferyl-glucuronide (MUG). The reaction with X-Glue generates a blue color that is useful in histochemical detection of the gene activity. For quantification purposes, MUG is preferred, because the umbelliferyl radical emits fluorescence under UV stimulation, thus providing better sensitivity and easy measurement by fluorometry (Jefferson et al., “GUS Fusions: μGlucuronidase as a Sensitive and Versatile Gene Fusion Marker in Higher Plants,” EMBO Journal 6:3901-3907 (1987), which is hereby incorporated by reference).

Other suitable selection markers include, without limitation, markers encoding for antibiotic resistance, such as the nptII gene which confers kanamycin resistance (Fraley, et al., Proc. Natl. Acad. Sci. USA, 80:4803-4807 (1983), which is hereby incorporated by reference) and the dhfr gene, which confers resistance to methotrexate (Bourouis et al., EMBO J. 2:1099-1104 (1983), which is hereby incorporated by reference). A number of antibiotic-resistance markers are known in the art and others are continually being identified. Any known antibiotic-resistance marker can be used to transform and select transformed host cells in accordance with the present invention. Cells or tissues are grown on a selection medium containing an antibiotic, whereby generally only those transformants expressing the antibiotic resistance marker continue to grow. Similarly, enzymes providing for production of a compound identifiable by luminescence, such as luciferase, are useful. The selection marker employed will depend on the target species; for certain target species, different antibiotics, herbicide, or biosynthesis selection markers are preferred.

Once a recombinant plant cell or tissue has been obtained, it is possible to regenerate a full-grown plant therefrom. Means for regeneration vary from species to species of plant, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

Plant regeneration from cultured protoplasts is described in Evans, et al., Handbook of Plant Cell Cultures, Vol. 1: (MacMillan Publishing Co., New York, 1983); and Vasil I. R. (ed.), Cell Culture and Somatic Cell Genetics of Plants, Acad. Press, Orlando, Vol. I, 1984, and Vol. III (1986), which are hereby incorporated by reference.

It is known that practically all plants can be regenerated from cultured cells or tissues, including but not limited to, monocots such as Gramineae (e.g., grass, corn, grains, bamboo, sugar cane), Liliaceae (e.g., onion, garlic, asparagus, tulips, hyacinths, day lily, and aloes), Iridaceae (e.g., iris, gladioli, freesia, crocus, and watsonia), and Orchidacea (e.g., orchid), and dicots including Salicaceae (e.g., willow, and poplar), Ranunculaceae (e.g., Delphinium, Paeonia, Ranunculus, Anemone, Clematis, columbine, and marsh marigold), Magnoliaceae (e.g., tulip tree and Magnolia), Cruciferae (e.g., mustards, cabbage, cauliflower, broccoli, brussel sprouts, kale, kohlrabi, turnip, and radish), Rosaceae (e.g., strawberry, blackberry, peach, apple, pear, quince, cherry, almond, plum, apricot, and rose), Leguminosae (e.g., pea, bean, peanut, alfalfa, clover, vetch, redbud, broom, wisteria, lupine, black locust, and acacia), Malvaceae (e.g., cotton, okra, and mallow), Umbelliferae (e.g., carrot, parsley, parsnips, and hemlock), Labiatae (e.g., mint, peppermints, spearmint, thyme, sage, and lavender), Solanaceae (e.g., potato, tomato, pepper, eggplant, and Petunia), Cucurbitaceae (e.g., melon, squash, pumpkin, and cucumber), Compositae (e.g., sunflower, endive, artichoke, lettuce, safflower, aster, marigold, dandelions, sage brush, Dalia, Chrysanthemum, and Zinnia), and Rubiaceae (e.g., coffee).

After the DNA construct is stably incorporated in transgenic plants, it can be transferred to other plants by sexual crossing or by preparing cultivars. With respect to sexual crossing, any of a number of standard breeding techniques can be used depending upon the species to be crossed. Cultivars can be propagated in accord with common agricultural procedures known to those in the field. Alternatively, transgenic seeds are recovered from the transgenic plants. The seeds can then be planted in the soil and cultivated using conventional procedures to produce transgenic plants.

Another aspect of the present invention is a method promoting yield from plants. This involves transforming a plant or plant seed with a nucleic acid of the present invention encoding a chitobiosidase, an endochitinase, or combination thereof, and regenerating the transformed plant to full grown as described above. Alternatively, transgenic seeds are recovered from the transgenic plants. The seeds can then be planted in the soil and cultivated using conventional procedures to produce transgenic plants.

Another aspect of the present invention is a method of reducing plant size. This involves transforming a plant or plant seed with a nucleic acid of the present invention encoding a chitobiosidase, an endochitinase, or combination t thereof, and regenerating the transformed plant to full grown as described above. Alternatively, transgenic seeds are recovered from the transgenic plants. The seeds can then be planted in the soil and cultivated using conventional procedures to produce transgenic plants.

Another aspect of the present invention is a method of reducing plant size. This involves transforming a plant or plant seed with a nucleic acid of the present invention encoding a chitobiosidase, an endochitinase, or combination thereof, and regenerating the transformed plant to full grown as described above. Alternatively, transgenic seeds are recovered from the transgenic plants. The seeds can then be planted in the soil and cultivated using conventional procedures to produce transgenic plants.

Confirmation of the presence of the transgene in transformed plants can be made by any of the methods for DNA analysis known or available to those skilled in the art, including, but not limited to, PCR and Southern blotting. Expression of chitinolytic activity, and other traits exhibited by the transgenic plants can be measured using standard assays known to those of ordinary skill in the art, and as described in further detail in the Examples below.

Just as the increased expression of chitinolytic genes promotes early flowering, increases yield, and reduces plant size in transgenics harboring one or more genes encoding for chitinolytic enzymes relative to control (non-transgenic) plants (see Examples, below), direct exposure of the chitinolytic enzymes of the present invention can be expected to affect those aspects of the development of plants so treated. Accordingly, the present invention also relates to a method promoting early flowering in plants by the direct application of a chitinolytic enzyme having either chitobiosidase or endochitinase activity. This method involves applying one or more isolated chitinolytic enzymes of the present invention to all or part of a plant or a plant seed under conditions effective to promote early flowering, increase yield and reduce plant size.

The method of the present invention involving application of a chitinolytic enzyme can be carried out through a variety of procedures when all or part of the plant is treated, including leaves, stems, roots, etc. This may (but need not) involve infiltration of the chitinolytic enzyme into the plant. Suitable application methods include topical application (e.g., high or low pressure spraying), injection, and leaf abrasion proximate to when enzyme application takes place. When treating plant seeds, in accordance with the application embodiment of the present invention, a chitinolytic enzyme can be applied by low or high pressure spraying, coating, immersion, or injection. Other suitable application procedures can be envisioned by those skilled in the art provided they are able to effect contact of the chitinolytic enzyme with the plant or plant seed. Once treated with a chitinolytic enzyme of the present invention, the seeds can be planted in natural or artificial soil and cultivated using conventional procedures to produce plants. After plants have been propagated from seeds treated in accordance with the present invention, the plants may be treated with one or more applications of a chitinolytic enzyme to promote early flowering, increase yield and reduce plant size.

Alternatively, the chitinolytic enzyme can be applied to plants, transgenic or non-transgenic plants, such that seeds recovered from such plants have been induced by the application of the chitinolytic enzyme to promote early flowering, or exhibit increased yield and/or reduce plant size.

The chitobiosidase or endochitinase can be applied to plants or plant seeds in accordance with the present invention individually, in combination with one another, or in a mixture with other materials. Alternatively, a chitinolytic enzyme can be applied separately to plants with other materials being applied at different times.

A chitinolytic enzyme of the present invention is preferably produced in purified form (preferably at least about 80%, more preferably 90%, pure) by conventional techniques. Typically, a chitinolytic enzyme of the present invention is secreted into the growth medium of recombinant host cells. Alternatively, a chitinolytic enzyme of the present invention is produced but not secreted into growth medium. In such cases, to isolate a chitinolytic enzyme, the host cell (e.g., E. coli) carrying a recombinant plasmid is propagated, lysed by sonication, heat, differential pressure, or chemical treatment, and the homogenate is centrifuged to remove bacterial debris. The supernatant is then subjected to sequential ammonium sulfate precipitation. The fraction containing a chitinolytic enzyme of the present invention is subjected to gel filtration in an appropriately sized dextran or polyacrylamide column to separate the proteins. If necessary, the protein fraction may be further purified by HPLC (U.S. Pat. No. 6,069,299 to Broadway et al., which is hereby incorporated by reference).

A composition suitable for treating plants or plant seeds in accordance with the application embodiment of the present invention contains a chitinolytic enzyme in a carrier. Suitable carriers include water, aqueous solutions, slurries, or dry powders. In this embodiment, the composition may contain greater than 500 nM chitinolytic enzyme.

Although not required, this composition may contain additional additives including fertilizer, insecticide, fungicide, nematicide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. These materials can be used to facilitate the process of the present invention. In addition, a chitinolytic enzyme can be applied to plant seeds with other conventional seed formulation and treatment materials, including clays and polysaccharides.

The present invention also relates to method of promoting increased yield by the application of a chitinolytic enzyme having either chitobiosidase or endochitinase activity. This method involves applying one or more isolated chitinolytic enzymes of the present invention to all or part of a plant or a plant seed, as described above, under conditions effective to promote increased yield.

The present invention also relates to method of reducing plant size by the application of a chitinolytic enzyme having either chitobiosidase or endochitinase activity. This method involves applying one or more isolated chitinolytic enzymes of the present invention to all or part of a plant or a plant seed, as described above, under conditions effective to reduce plant size.

### EXAMPLES

### Example 1

### Cloning, Transformation and Verification by PCR

Cloning of the endochitinase and chitobiosidase genes from S. albidoflavus was carried out as follows. The chitobiosidase and endochitinase genes, corresponding to SEQ. ID. Nos. 2 and 4, were obtained from Dr. David Williams, Cornell University, Ithaca, N.Y. Each DNA was cloned individually into the plasmid pBluescript (Stratagene, LaJolla, Calif.), and amplified using synthetic PCR primers designed with restriction sites to allow subcloning into the appropriate sites in pBI525, a plasmid which contains a CaMV35S promoter and tandem elements of upstream enhancer elements that increases the expression of protein more than 10-fold (Kay et al., “Duplication of CaMV35 S Promoter Sequences Creates A Strong Enhancer for Plant Genes,” Science 236, 1299-1302 (1987), which is hereby incorporated by reference). In order to determine that the genes were each cloned and in frame, a modified mini alkaline-lysine/PEG precipitation procedure was used to isolate plasmid DNA (Tartof et al., “Improved Media for Growing Plasmid and Cosmid Clones,” Bethesda Research Laboratory Focus 9:12 (1987), which is hereby incorporated by reference), followed by DNA sequencing. Automated DNA sequencing was performed using the ABI PRISM Ready Reaction DyeDeoxy Terminator Cycle Sequencing Kit (Perkin-Elmer, Foster City, Calif.).

The expression cassette from 1 μg of pBI525 was excised with HindIII and EcoRI and inserted into the multicloning site of the binary vector pBin19, resulting in the transformation plasmids pS. a-chitobiosidase (containing the chitobiosidase S. albidoflavus sequence) and pS. a-endochitinase containing the endochitinase S. albidoflavus sequence). A third transformation plasmid pS.a-endochitinase-chitobiosidase, containing both the S. albidoflavus endochitinase and chitobiosidase sequences in a single expression cassette, was created using the same method.

The pS. a-endochitinase chitobiosidase transformation plasmid was introduced into A. tumefaciens, which was used to transfer the genes into tomato (Lycopersicon esculentum), using a modification of McCormick et al., “Leaf Disc Transformation of Cultivated Tomato (L. esculentum), using Agrobacterium tumefaciens,” Plant Cell Reports 5::81-84 (1986), which is hereby incorporated by reference, as reported by Xue et al., “Development of Transgenic Tomato Expressing a High Level of Resistance to Cucumber Mosaic virus Strains of Subgroups I and II,” Plant Disease 78:1038-1041 (1994), which is hereby incorporated by reference.

Four tomato cultivars were used for the transformation: Lycopersicon esculentum UC82B, Better Boy VFN, Beefmaster VFN (“Bm”) and Geneva 80. Following transformation and regeneration, the tomato plants were transferred to soil, and allowed to grow until they produced fruit, and the seed was collected. The initial transformants which harbored at least one copy of the transgene are referred to herein as the “T0” plants. T1 plants are those grown from the seed collected from the T0 plants, and T2 are transgenic plants grown from the seed of the T1 plants. The T1 and T2 transgenic tomato plants which showed the highest level of chitinolytic activity were Beefmaster lines BmB1-1, and BmB1-7, which were used for subsequent testing.

Prior to the use of the tomato plants for determination of chitinolytic activity, the presence of the nptII marker gene was determined by PCR amplification, using standard PCR with a cycle of 94° C.×5 min 1 cycle, 94° C.×30 sec, 67° C.×30 sec, 72° C.×1 min 30 cycles, and 72° C.×5 min 1 cycle, and primers designed to amplify a region of the T-DNA containing the nptII gene. Transgenic plants that were nptII negative were discarded.

### Example 2

### T2 Tomato lines BMB1-1/7: Endochitinase and Chitobiosidase Activity

Young, fully developed leaflets from the first leave of control (non-transgenic) Beefmaster tomato plants and T2 transgenic (Beefmaster) tomato plants (Bm 1-1/7) transformed with pS.a-endochitinase-chitobiosidase as described in Example 1, and expressing both the endochitinase and chitobiosidase genes from Streptomyces albidoflavus were evaluated for endochitinase and chitobiosidase activity at 30 and 45 days post-seed planting. The determination of the activity was performed on these days, because, at 30 days, the seedlings were ready to be transplanted to individual pots, and, at 45 days, the transgenic plants initiated the formation of the flowering buds.

Levels of endochitinase and chitobiosidase expression were quantified by enzymatic activity assays using methyl umbelliferyl substrates. Activity slopes for the methyl umbelliferyl enzymatic reaction were determined for each sample (nM MU/min), and the value of nM MU/min was corrected for the amount of protein (nM/min/μg protein).

Two different sets of plants were used for enzyme activity correlation analyses. Set I included 20 controls (non-transgenic Beefmaster tomato plants) and 20 T2 BmB1-1 transgenic plants. Set II consisted of 20 controls and 30 BmB1-1 transgenic plants. In addition to the 30 and 45 days post-planting analysis of enzyme activity, chitinolytic activity was measured in Set I at 105 days post-seed planting (after transgenic plants have produced fruits), while the third measurement for Set II was done at 75 days post-seed planting (when transgenic plant were producing fruits and controls were just at flowering).

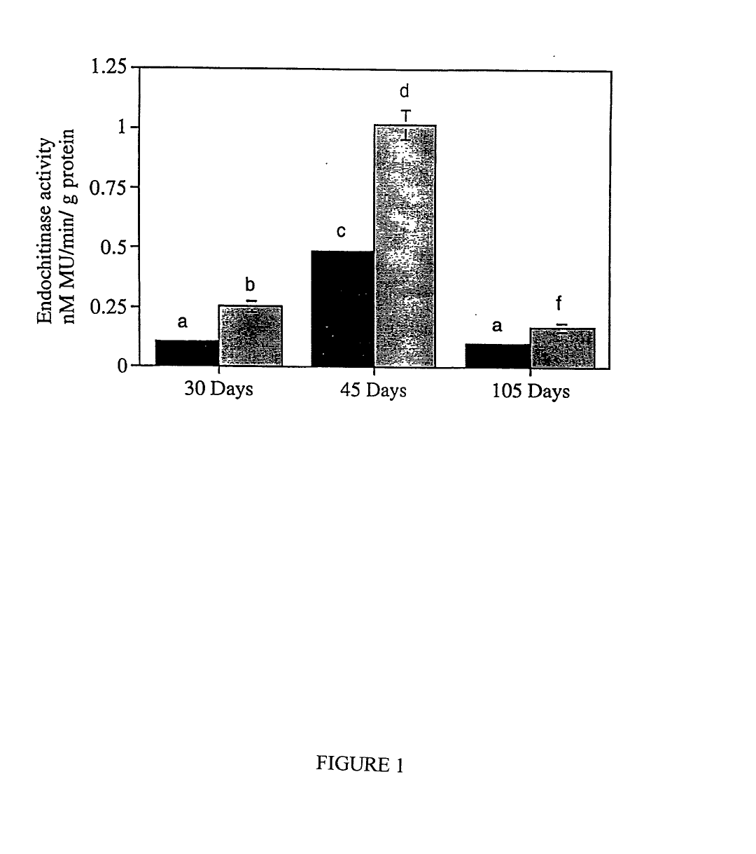

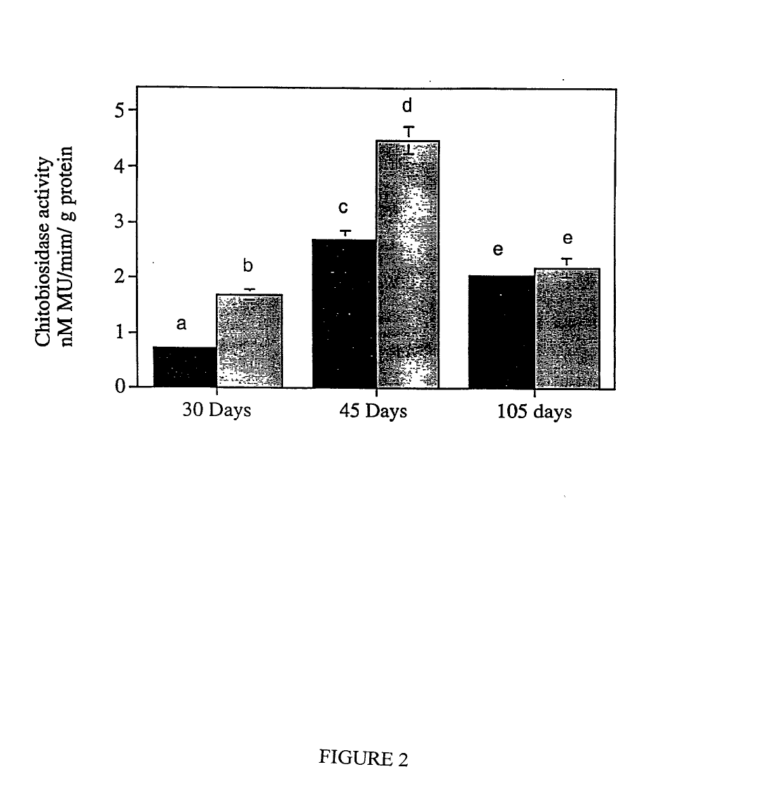

Results of endochitinase activity over time for Set I are shown in FIG. 1. FIG. 2 shows the chitobiosidase activity over time for Set I. The ANOVA analysis indicated that for all the three time intervals, endochitinase activity was significantly different between control and transgenic plants [30 days (F=28.78, p<0.001), at 45 days (F=38.52, p<0.001), and 105 days (F=4.95, p=0.035)]. For chitobiosidase activity, the difference was significant between control and transgenic plants at 30 days (F=62.74, p<0.001) and 45 days (F=28.13, P<0.0001) but not at 105 days (F=0.34, p=0.564).

When the levels of activity of both endochitinase and chitobiosidase in transgenic plants was compared over time, the difference was significant [i.e., for endochitinase 30 vs. 45 days (F=134, P<0.001) and 45 vs. 105 (F=161, p<0.001)1]. The control plants showed the same response [i.e., for endochitinase activity 30 vs. 45 days (F=130, p<0.001) and 45 vs. 105 (F=88.28, p<0.001)1.]

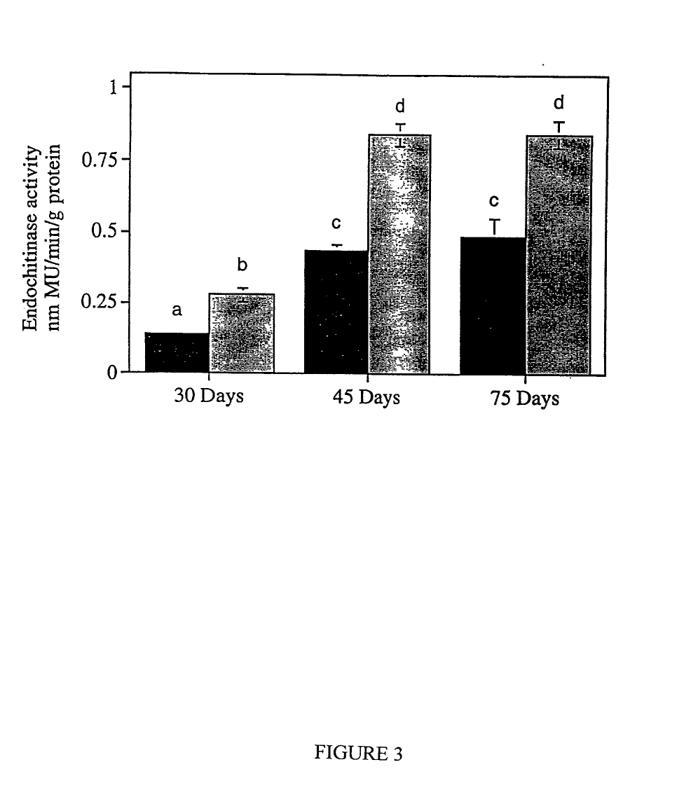

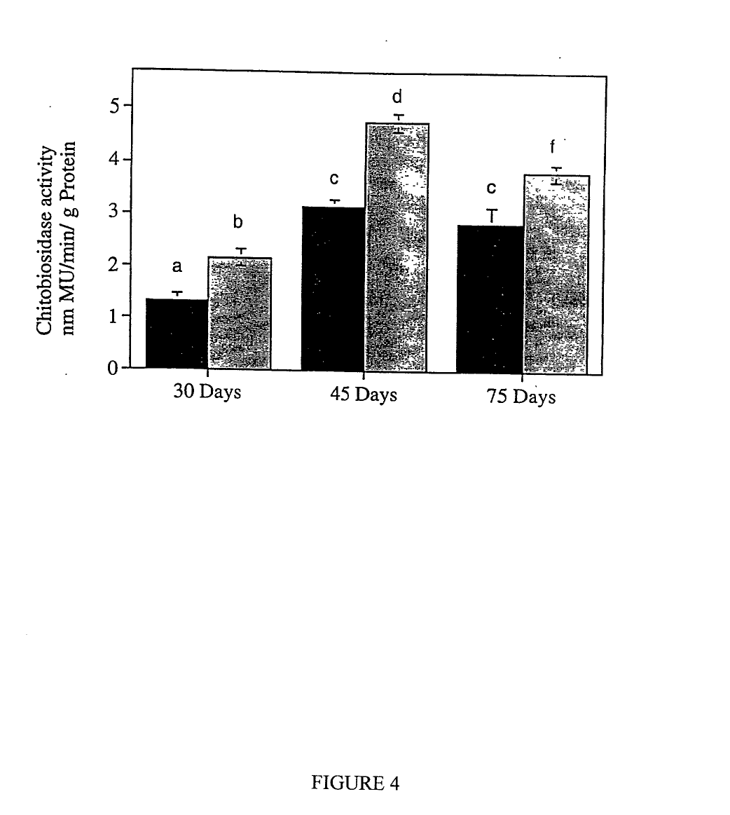

Results of endochitinase activity over time for Set II are shown in FIG. 3. Chitobiosidase activity over time for Set II is shown in FIG. 4. ANOVA analysis indicated that at all three time intervals, the difference was significant between control and transgenic plants for both endochitinase activity and chitobiosidase. For the endochitinase activity, at 30 days F=18.4, p<0.001, at 45 days F=41.17, p<0.001 and at 75 days F=14.48, P=0.035. Similar results were obtained for the chitobiosidase activity.

In the transgenic plants, when the enzyme activity was compared at different times, there was a difference in the endochitinase activity when comparing 30 days and 45 days (F=73.65, p<0.001), but no difference was found between 45 and 75 days. In the case of the chitobiosidase activity, there was a significant difference at all three times (F=71.94, p<0.001). For the control plants, the difference was significant between 30 and 45 days for both endochitinase activity and chitobiosidase activity. There was a difference between 30 and 45 days for endochitinase activity (F=57.05, p<0.001) and for chitobiosidase activity (F=38.50, p<0.001). However, no difference was found between 45 and 75 days.

### Example 3

### T2 Tomato lines BMB1-1/7: Endochitinase and Chitobiosidase Activity vs. Plant Size

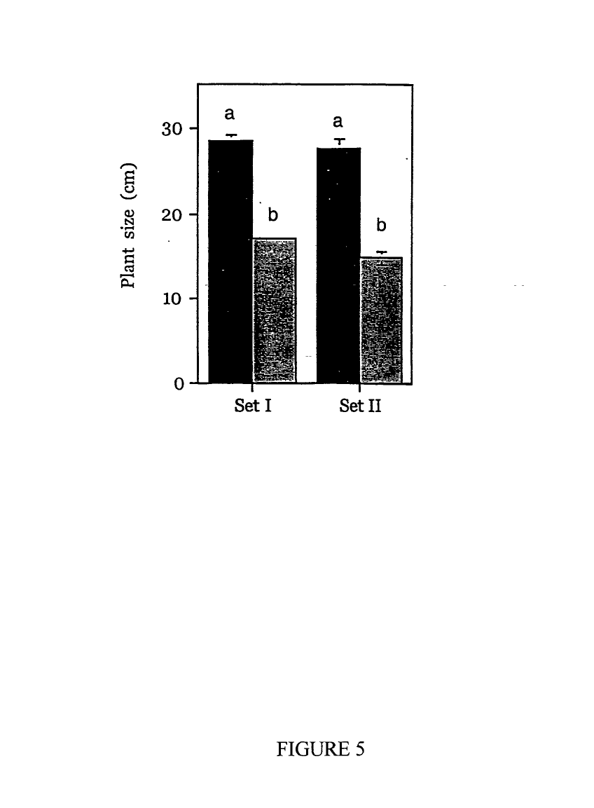

Plant height (cm) was determined 45 days post-seed planting for both Set I and Set II, as shown in FIG. 5. ANOVA analysis indicated that there was a significant difference between the height of the control plants and the height of the transgenic plants [Set I F=159.0, p<0.001; Set II F=95.57, p<0.001].

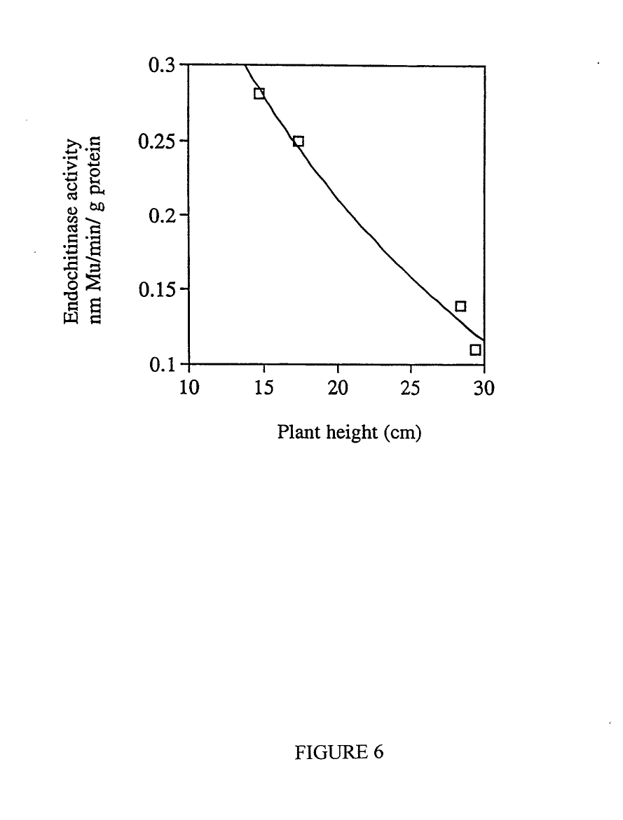

The endochitinolytic activity and chitobiosidase activity measured at 30 days post-planting was correlated with the height of the plants measured 35 days post-planting (r2=0.989, Y=−0.546 log (x)+0.923 for endochitinase activity, shown in FIG. 6, r2=0.833, Y=−3.63 log W+6.307 for chitobiosidase activity). Example 4

### T2 Tomato lines BMB1-1/7: Endochitinase and Chitobiosidase Activity vs. Flowering Time

Flowering time was measured by counting the days from transplanting to the formation of the first flowering buds. The endochitinase and chitobiosidase activities measured at 45 days post-seed planting were correlated with the flowering time (r2=1.0, Y=−1.889 log (x)+3.366 for endochitinase activity, r2=0.823, Y=−6−744 log W+13.207) for chitobiosidase activity). The flowering time for the control plants was significantly different from transgenic plants, for set I (F=36.68, p<0.001), as shown in FIG. 6. For Set II, the transgenic plants flowered at 21.23±0.65 days post-transplanting. However, the control plants did not flower after 30 days post-transplanting.

### Example 5

### Identification of T1 Transgenic Lines

In order to eliminate the possibility that the effect on growth and flowering time observed in the T2 transgenic line was due to a transgene position effect, rather than the expression of the endochitinase and chitobiosidase transgenes, ten seeds from 10 different T0 transgenic plants that were transformed with the same construct (pS.α-endochitinase-chitobiosidase) as the T2 plants were planted.

DNA was isolated from the leaves of the T1 tomato plants (20 day old plants, with 2 pairs of leaves) growing in the greenhouse, using the minipreparation method described by Cheung et al., “A Simple and Rapid DNA Microextraction Method for Plant, Animal, and Insect Suitable for RAPD and other PCR Analysis,” PCR Methods Appl. 3, 69-70., et al. (1993), which is hereby incorporated by reference. A pair of primers was chosen to amplify a region of the T-DNA containing the nptII gene, using a standard PCR reaction with a PCR profile as follows: 94° C.×5 min 1 cycle, 94° C.×30 sec, 67° C.×30 sec, 72° C.×1 min 30 cycles, 72° C.×5 min 1 cycle.

Ten seeds from each of the ten different T0 lines and 12 seeds from control plants, identified below in Table 1, were planted. PCR was performed when the plants germinated using a leaflet for the first true leaf to identify the presence of nptII gene. Plants that were nptII negative were discarded. Table 1 shows the number of seeds germinated from each line and the number of plants that were nptII positive.

TABLE 1
Tomato T1 lines nptII PCR results

| | # seeds | # Plants |
|---|---|---|
| Tomato Lines | germinated | PCR nptII positive |
| B1 | 6 | 4 |
| C1 | 6 | 4 |
| D1 | 10 | 8 |
| F1 | 10 | 7 |
| F2 | 8 | 6 |
| F3 | 10 | 8 |
| F4 | 10 | 3 |
| H1 | 3 | 2 |
| H2 | 7 | 6 |
| J1 | 10 | 0 |
| Controls | 12 | 0 |

The plants that were identified as nptII positive were transplanted individually into soil in pots, and Southern blot analyses were performed on one plant from each line to determine if the complete T-DNA fragment was inserted, and to determine the number of copies of transgene per genome.

Southern analyses were performed on the T1 plants to determine the copy number of the T-DNA, endochitinase gene and chitobiosidase gene.

DNA was isolated from leaflets from the plants growing in the greenhouse using a modification of the miniprep technique described by Fulton et al., “Microprep Protocol for Extraction of DNA from Tomato and Other Herbaceous Plants,” Plant Mol. Bio. Rep. 13, 207-209 (1995), which is hereby incorporated by reference. For restriction enzyme digestion of genomic DNA, 10-15 pg of genomic DNA was incubated with 100 U of the restriction enzyme HindIII, and 5 μl of buffer, in a final reaction volume of 50 μl, at 37° C., overnight. Random oligonucleotide primers (Sambrook et al., Molecular Cloning: A Laboratory Manual, Second Edition, Cold Spring Harbor Press, NY (1989), which is hereby incorporated by reference) were used for radioactive labeling of the probes using the T-DNA from the pS.α-endochitinase chitobiosidase as templates, the probe for the chitobiosidase gene was designed using the chitobiosidase gene inserted in the plasmid pBluescript II SK (Stratagene, La Jolla, Calif.). The probe for the endochitinase gene was designed using the endochitinase gene excised with Nco and Xba from pS.α-endochitinase plasmid.

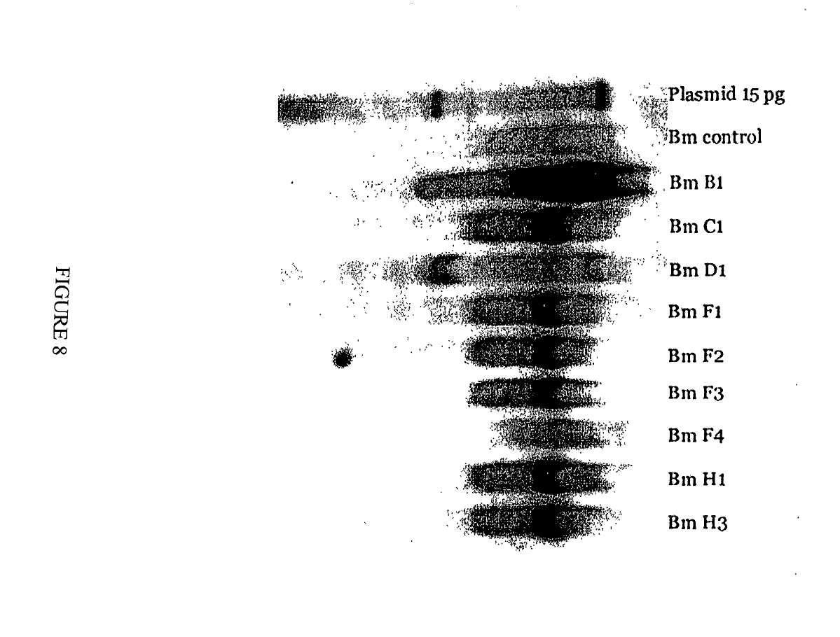

FIG. 8 shows the Southern blot using a T-DNA probe synthesized from the construct pS.αendochitinase-chitobiosidase to evaluate the lines of tomato. Line B1 showed 5 copies of the T-DNA, lines C1, D1, F1, F2, F3 H1 and H3 showed 2 copies, and line F4 was not transformed.

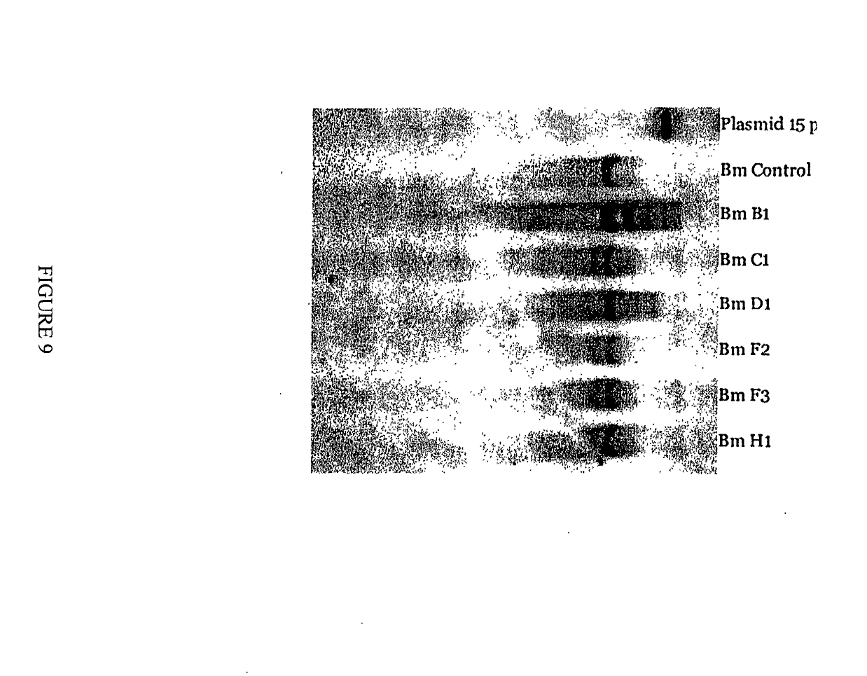

FIG. 9 shows the Southern blot, using the chitobiosidase gene as a probe (synthesized from the construct pBS-chitobiosidase) for the lines of tomato. The lane that corresponds to the control showed a band approximately 5 kb. The same band was observed in all the other lines, which indicated that it may correspond to none specific binding or a contamination. However, in addition to this band, line B1 showed 3 copies of the genes with molecular weight higher than 5 kb, line D1 showed 1 copy with molecular weight higher than 5 kb and, lines C1, F2, F3 and H1 showed 1 copy with a molecular weight lower than 5 kb.

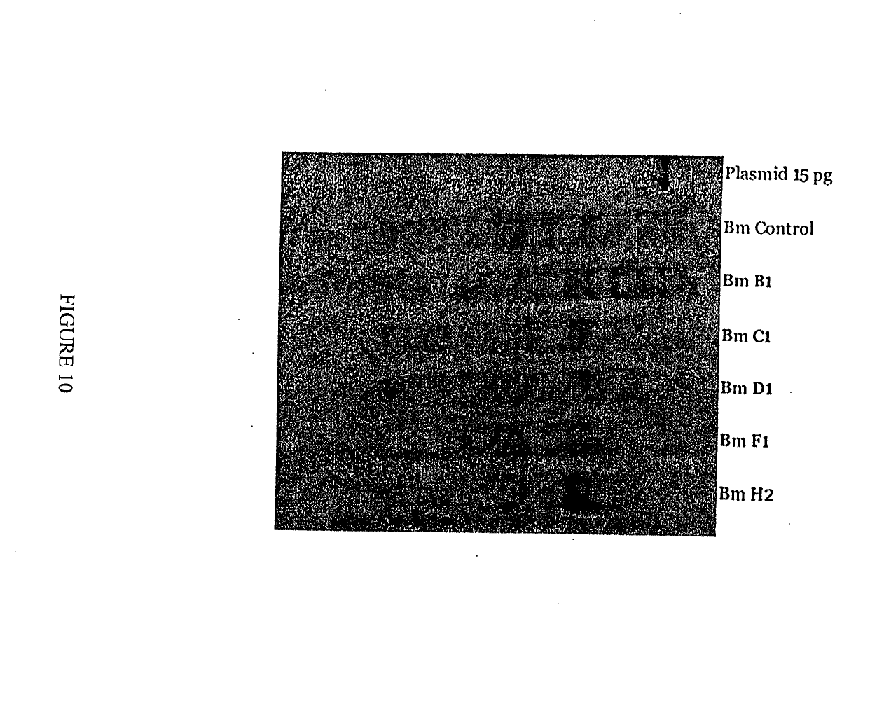

FIG. 10 shows the Southern blot using the endochitinase gene as a probe (synthesized from the endochitinase gene that was excised with Nco and Xba from pS.α-endochitinase plasmid) for the lines of tomato. The lane that corresponds to the control plants showed a band around 5 kb. The same band was observed in all the other lines, which indicated that it may correspond to nonspecific binding or a contamination. However, in addition to this band, line B1 showed 3 copies of the genes with molecular weight higher than 5 kb, lines C1, F1 and H2 showed 1 copy with molecular weight lower than 5 kb, and line D1 showed no copies of the gene. Although Line D1 did not contain the endochitinase gene, it did contain the chitobiosidase gene.

### Example 6

### Endochitinase and Chitobiosidase Activity vs. Plant Height in Different T1 Tomato Lines

After determining that the T1 lines contained the endochitinase and chitobiosidase transgenes, fully developed leaflets from the first leaves of the plants were evaluated for endochitinase and chitobiosidase activity at 30 and 45 days post-seed planting. The height of each of the plant was measured 45 days post-seed planting, the number of leaves in each plant was counted, and the time from transplanting to flowering was noted.

### Example 7

### Endochitinase and Chitobiosidase Activity vs. Plant Height in Different T1 Tomato Lines

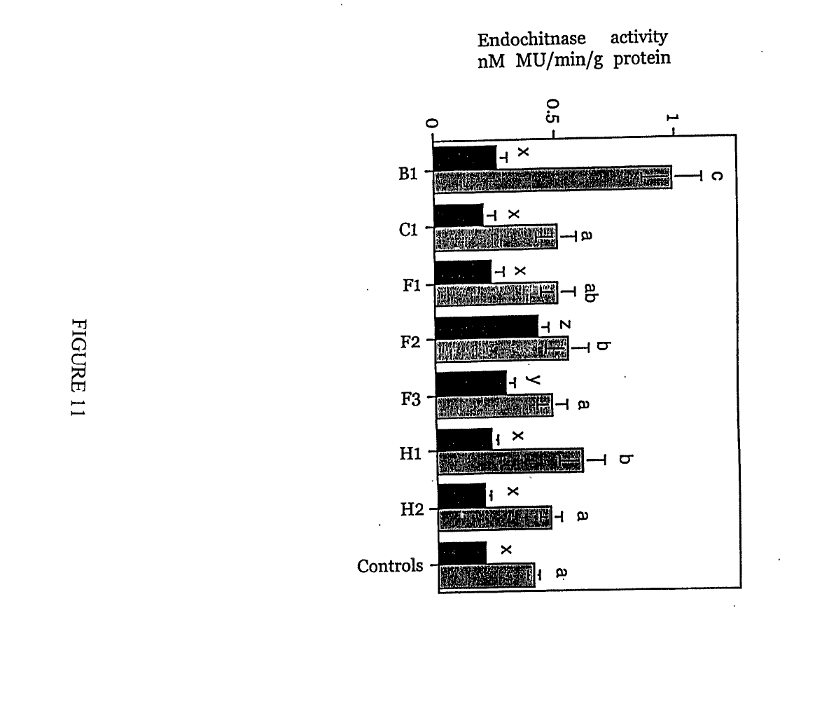

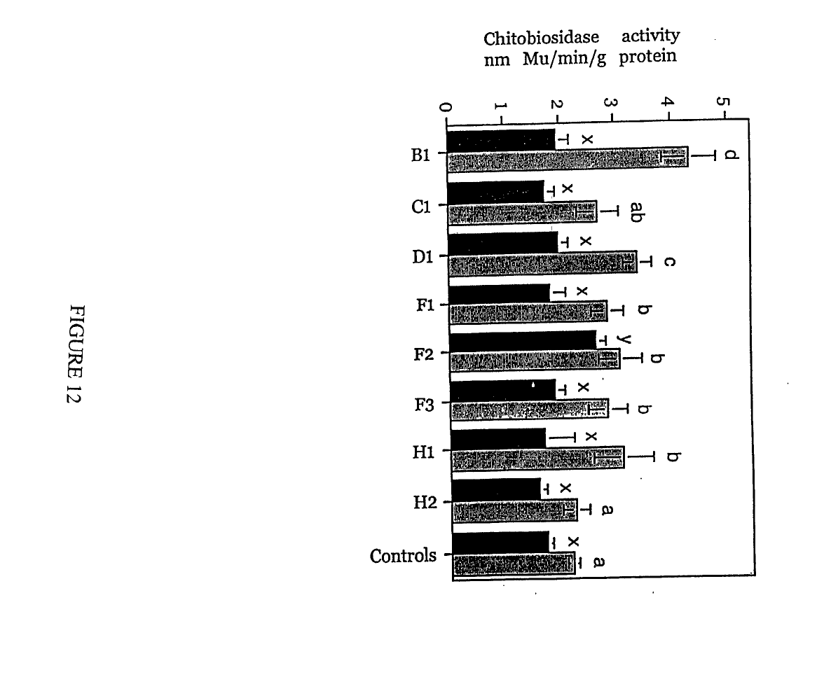

All the T1 plants that were nptII positive were evaluated for endochitinase and chitobiosidase activity at 30 days post-seed planting, which was just prior to transplanting to large pots. The plants were allowed to grow for an additional 15 days (45 days post-seed planting) and, at this point, the height of the plants was measured. In addition, the endochitinase and chitobiosidase activity was determined at 45 days post-seed planting, at which time the transgenics started to flowering. FIG. 11 and FIG. 12 show the endochitinase and chitobiosidase activity, respectively, over time in the T1 plants. Table 2 shows the statistical differences (ANOVA) for endochitinase and chitobiosidase activity between the T1 lines and control plants at 45 days post-planting. Lines B1, F2 and H1 differed from the control for the endochitinase and chitobiosidase activity. Lines D1, F1, and F3 differed from the control for chitobiosidase activity but not for endochitinase (line D1 did not show the endochitinase gene in the Southern blot) and lines C1 and H2 did not differ from the controls for endochitinase nor endochitinase activity.

TABLE 2
ANOVA differences between tomato TI transgenic lines and control
plants respect to endochitinase activity and chitobiosidase activity.

| | ANOVA | | | | | |
|---|---|---|---|---|---|---|
| | Endochitinase | | | ANOVA | | |
| | activity | | | Chitobiosidase activity | | |
| Line | Control vs. Line | | | Controls vs. Lines | | |
| | F | p | | F | p | |
| B1 | 58.83 | <0.001 | c | 46.78 | <0.001 | d |
| C1 | 2.85 | 0.11 | a | 3.06 | 0.111 | ab |
| D1 | | | | 25.21 | <0.001 | c |
| F1 | 3.50 | 0.075 | ab | 6.41 | 0.019 | b |
| F2 | 5.07 | 0.036 | b | 8.97 | 0.007 | b |
| F3 | 2.53 | 0.127 | a | 5.35 | 0.03 | b |
| H1 | 6.89 | 0.018 | b | 7.24 | 0.016 | b |
| H2 | 1.68 | 0.211 | a | 0.07 | 0.789 | a |
| Control | | | a | | | a |

Note: a, b, c, d: lines associated with a different letter are significantly different.

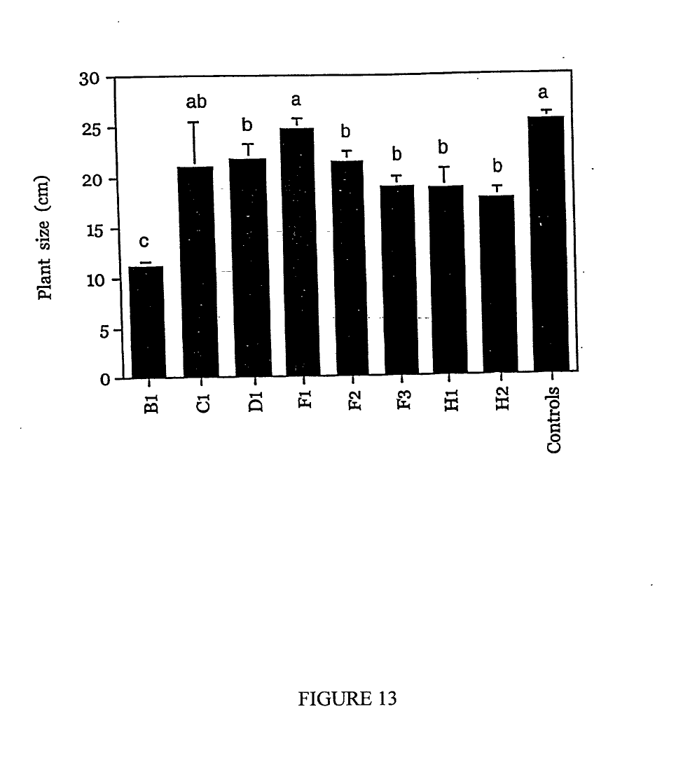

When the size of the plants was measured at 45 days post-seed planting, no difference was found between the controls and lines C1 and F1, as shown in FIG. 13. In all the other lines, the difference was significant with respect to the controls (F=6.5, p=0.001). No difference was found between the number of leaves in the controls and the transgenic lines.

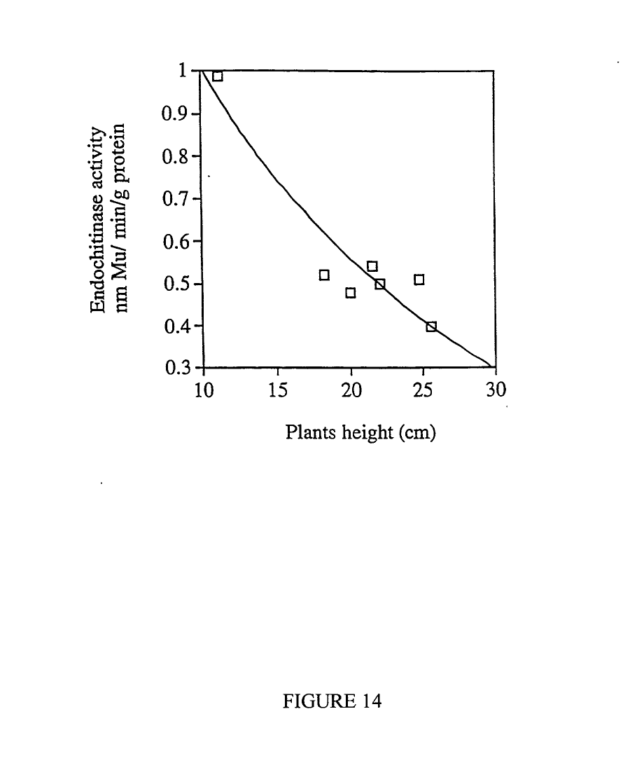

At 45 days post-seed planting, there was a negative correlation between height of the plants and chitinolytic enzymes, shown for endochitinase in FIG. 14 (r2=0.785, Y=−1443 log(x)+2.42-3), F=13.76, p=0.010)1, and for chitobiosidase activity (r2=0.541, Y=−4.3 log W+8.54), F=5.91, p=0.0451.

### Example 8

### Endochitinase and Chitobiosidase Activity vs. Flowering Time in Different T1 Tomato Lines

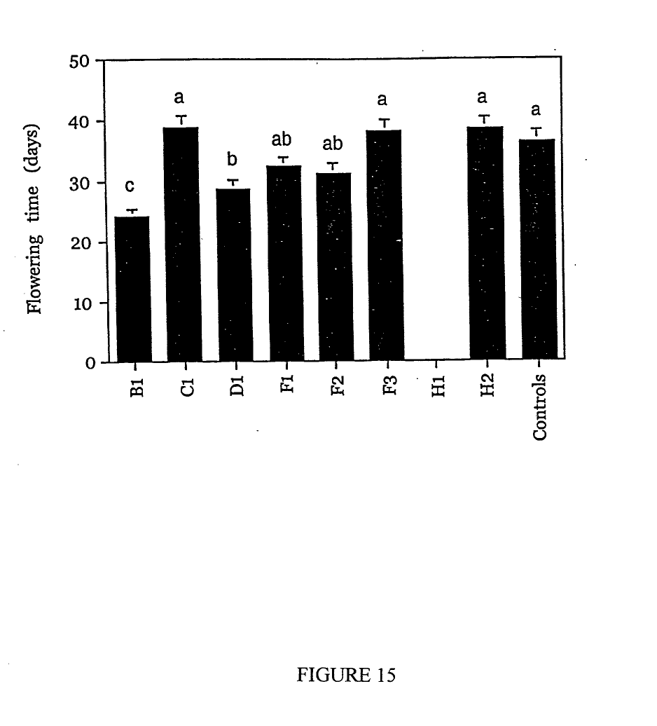

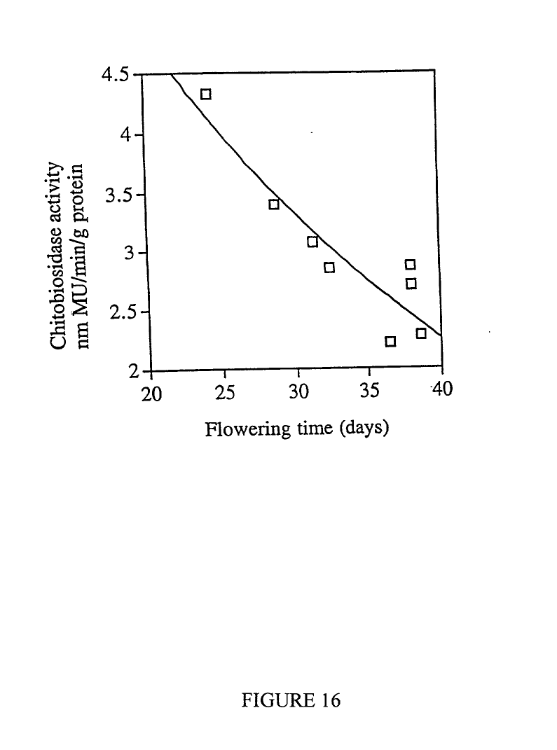

When the flowering time post-transplanting (measured in days) in the controls was compare with the transgenic T1 lines, only the lines B1 and D1 differed significantly from the controls (F=9.8, P=0.001), as shown in FIG. 15. At 45 days post-seed planting, there was a negative correlation between days to flowering and chitinolytic enzymes. For endochitinase (r2=0.807 (Y=−2.374 log(x)+4.18), F=15.13, p=0.012). For chitobiosidase, shown in FIG. 16, (r2=0.832, Y=−8-44 log (x)+15.8), F=23.62, P=0.0031.

### Example 9

### Effect of Leaf Trimming on T2 Transgenic Tomato Line

Between 10 and 20 control plants and T2 BmB1-1 were trimmed by manually breaking off the lateral leaves when they started to grow (“trimmed”). Another set of plants (control and transgenic) planted at the same time was allowed to grow without leaf removal (“non-trimmed”). In the 2 treatments, the flowering time post-transplanting and the number of fruits produced on the plants 60 days post-transplanting were noted.

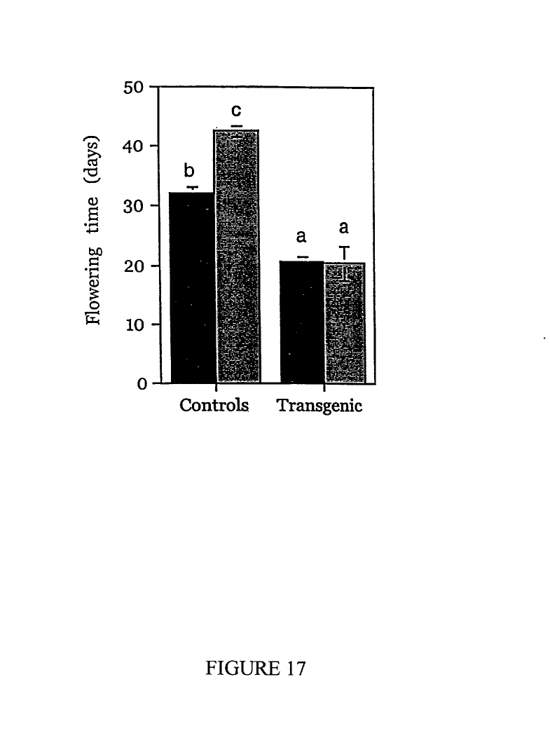

With respect to the flowering time, in both the trimmed and non-trimmed groups of plants, the control plants differed significantly from the transgenic plants, as shown in FIG. 17. For trimmed plants F=82.65, p<0.001; for non-trimmed plants F=92.55, p<0.001). When comparing the two sets of control plants, the non-trimmed plants were significantly different from the trimmed plants (F=9.7, p=0.007). The control plants that were trimmed flowered in less time (11 days sooner) than the non-trimmed control. However, the transgenic plants that were trimmed did not differ from the non-trimmed transgenics (F=0.04, p=0.838).

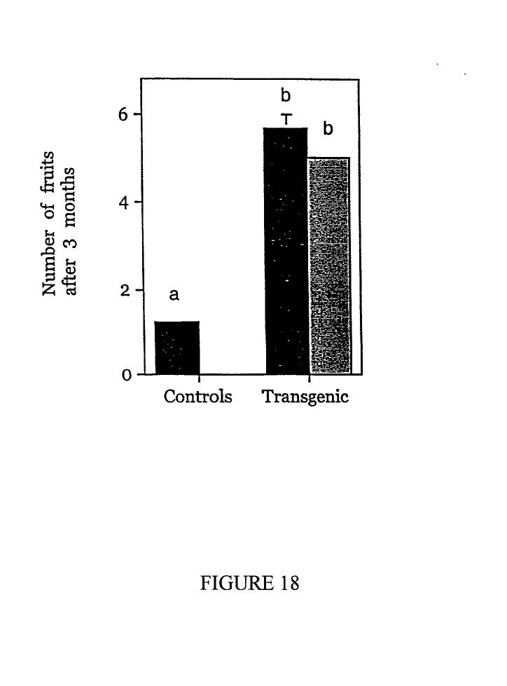

FIG. 18 shows the effect of trimming on fruit production. Non-trimmed control plants did not produce fruit after 60 days post-planting. In the group of plants that was trimmed, the control plants produced significantly less fruit than the transgenic plants(F=32.4, p<0,001). The fruit production on the transgenic plants that were trimmed did not differ from the non-trimmed transgenic.

These findings suggest that chitinolytic enzymes have an important role in the development and flowering time of tomato plants. Independent of the fact that the plants were transformed with the transgenes from S. albidoflavus, in all cases, there was an increase of endochitinase and chitobiosidase activity from 30 to 45 days post-seed planting. This suggests that there is an accumulation and/or induction in production of the chitinolytic enzymes during normal development of the plants.

Plants that were evaluated for chitinolytic enzyme activity after 45 days (T2 plants and controls) indicated two important factors: Firstly, during the normal development of the plants, there was an increase in the level of chitinolytic activity in the leaves from the time of transplanting (30 days after seed planting) until the plants produced the flowering buds. After that, the levels of enzymes remained constant until the fruit matured. Then, the levels decreased significantly, as seen in FIGS. 1-4. Secondly, the formation of flowering buds in the transgenic plants is produced at least 15 days earlier than in the control plants.

The analyses of the different T1 lines transformed with the same construct as the T2 BmB1 line indicated that the enhanced growth and early flowering bud formation following transgenic insertion of the bacterial endochitinase and chitobiosidase was not result of the insertion position of the transgenes. The Southern blots showed that most of the T1 lines were transformed with both genes (endochitinase and chitobiosidase). When the Southern blots were probed with the endochitinase or chitobiosidase gene, a band around 5 Kb was observed in the control and the transgenic lines. This band may correspond to non-specific binding, contamination, or it may be the result of homology with some sequence in the tomato genome. However, sequence homology between the chitinolytic genes from S. albidoflavus and plant genes have not been reported before, and no consensus sequences were found between those genes and plant genes when an analysis of homology was performed using the data bank “GenBank” (National Center of Biotechnology Information NCBI). Although the T1 lines showed the 5 kb band, in addition, they also showed other bands that corresponded to the chitinolytic enzymes. When the T1 lines were analyzed for chitinolytic activity, different levels of the enzymes were observed in the different lines, and when the levels of the enzyme activity were evaluated in relation to the height of the plants and the flowering time, a high correlation was observed, as shown in FIG. 15 and FIG. 16. This suggests that the effect of the chitinolytic enzymes in the development of the tomato plants is a common characteristic that may be observed in all the tomato lines transformed with the endochitinase and/or chitobiosidase from S. albidoflavus.

Based on these findings, it is believed that, for the tomato plants to initiate the process of formation of flowering buds, a threshold in the level of endochitinases and/or chitobiosidases have to be reach in the plant, and this threshold will trigger the formation of the flowering buds in the plants. It is known that the morphogenic changes evident in plants during the transition from vegetative to reproductive development are accompanied by the appearance of new gene products (Pierard et al., “Appearance and Disappearance of Proteins in the Shoot Apical Meristem of Sinapis alba in Transition to Flowering,” Planta 150:397-405 (1980)). It is believed that chitinolytic enzymes are part of this group of genes that are induced prior to flowering.

In the experiments performed with the T2 transgenic plants and controls, the transgenic plants contained higher levels of endochitinase and chitobiosidase than the control. Therefore, the threshold of chitinolytic enzymes, that induced the initiation of flowering was reached in a shorter time in the transgenic plants than in the controls. The transgenic plants initiated the process of bud formation almost 15 days prior to the control plants. It is believed that both enzymes are involve in the process of early flowering. When the levels of endochitinase and chitobiosidase in the T2 transgenic and control plants were compared with the time of flowering, high correlation was obtained with both enzymes (for the endochitinase levels r2=1 and for the chitobiosidase r2=0.82) and when the levels of chitobiosidase in the T1 transgenic plants and controls was correlated with the time of flowering, a correlation with r2=0.83 (F=23.62, P=0.003) was found, and the one observed with the endochitinase gene was r2=0.807 (F=15.13, p=0.012).

The above experiments also show that the chitinolytic enzymes have an effect on the height of the plants. In the T2 lines, when the transgenic plants were measured 30 days post-seed planting, the plants were shorter than the control plants. In the case of the T1 lines, measured 45 days post-seed planting, differences in height between transgenic and control plants also was observed. However those differences were smaller than at 30 days. After flowering, the differences in height when comparing control and T2 or T1 lines were not significant. It seems that the differences in plant height were observed only when the plants were young (i.e., before flowering), and the effect on height was more related with the levels of endochitinase than chitobiosidase. When a correlation between the plant height and endochitinase levels was determined for the different T1 lines, the r2=0.78 with F=13.76, p=0.010, while the correlation with the chitobiosidase levels was smaller (r2=0.541. F=5.91, p=0.045). A high correlation with both enzymes was observed in the T2 lines, but this can be the result of the high levels (up to 5 fold higher) of chitinolytic activity that the transgenic plants showed compared to the controls. The levels of chitobiosidase activity in the T1 lines were lower compared with the T2 lines. In addition, when the endochitinase gene from S. albidoflavus was used to produce apple transgenic plants, the plants that were regenerated in tissue culture failed to become established in soil, a phenomenon that did not take place in apples transformed with the chitobiosidase gene. This deleterious effect in plant growth was also observed in apple plants transformed with another endochitinase gene from Trichoderma harzianum. In this experiment, the levels of endochitinase activity in the apple plants were more than 10 fold higher than the ones observed in the tomato plants.

In contrast, Patil et al., “Possible Correlation Between Increased Vigour and Chitinase Activity Expression in Tobacco,” Journal of Experimental Botany 48:1943-1950 (1997) found a positive relation between the vigor of the plants and the levels of endochitinase expressed by a transgenic tobacco transformed with an endochitinase (Ch2) isolated from Zea mays. The higher seedling-vigor in the transgenic progeny appeared to be correlated with the presence of the chitinase (Ch2). However, these differences were observed in 2 of 4 experiments, and, although that gene was used in numerous studies before, no other effect on growth was reported.

Clearly, the level of chitinolytic enzymes activity is linked to plant development. However, it is not known why chitinolytic enzyme activity increases prior to the flower bud formation, or what substrate is digested by the chitinolytic enzymes. Immunological studies showed the presence of the GlcNac residues in the secondary cell wall of plants (Benhamou et al., “Attempted Localisation of a Substrate for Chitinase in Plant Cell Reveals Abundant N-acetyl-D-glucosamine Residues in Secondary Wall,” Biol. Cell 67:341-350 (1989)). It is possible that the chitinolytic enzymes play a role in cell wall breakdown by hydrolyzing the GlcNac residues. Morphogenetic changes that occur during plant development necessitate disruption of existing plant tissues. Such changes may occur during pollen tube growth, the formation of various organs such as lateral or adventitious roots (Varner et al., “Plant Cell Wall Architecture,” Cell 56:231-239 (1989)), seed germination, leaf senescence, and, most important in our study, the transitions from vegetative to floral meristem (Neale et al., “Chitinase, β3-1,3-Glucanase, Osmotin, and Extensin are Expressed in Tobacco Explants; During Flower Formation,” Plant Cell 2:673-684 (1990)). The direct effect of chitinolytic enzymes on the degradation of the cell wall has been observed in barley seed. In the seeds, the chitinolytic enzyme are induced during inhibition, and they hydrolyze the wall of the aleurona cells of the endosperm that contains β-1-3-glucan, causing the digestion of the aleurona wall, and allowing the emergence of the radicule. (Vogeli-Lange et al., “Evidence for a Role of β-1,3-Glucanase in Dicot Seed Germination,” The Plant Journal 5:273-278 (1994)). It is believed that the formation of the bud flowers may involve a similar process.

With respect to the effect on the growth of the plants, it has been postulated that the action of plant growth regulators may be mediated via oligosaccharides released from plant cell Walls by hydrolytic enzymes (Cote et al., “Oligosaccharides: Structures and Signal Transduction,” Plant Molecular Biology 26:1379-1411 (1994)). If the chitinolytic enzymes can hydrolyze the GlcNac residues and promote the release of those oligosaccharides, they may be involved in signal transduction pathways for developmental events (Hanfrey et al., “Leaf Senescence in Brassica napus: Expression of Genes Encoding Pathogenesis-Related Proteins,” Plant Molecular Biology 30:597-609 (1996)). The action of the phytohormones may be mediated by oligosaccharides released from plant cell wall due to the effect of chitinolytic enzymes.

The Examples disclosed herein provide further indications that the role of the chitinolytic enzymes is not necessarily restricted to plant defense. It is clear that there is a correlation between early flowering and plant height with the levels of chitobiosidases and endochitinases in the tomato transgenic plants. It cannot be determined whether this effect is exclusively due to the expression of the transgenes of endochitinase and chitobiosidase from S. albidoflavus or the additive effect of these 2 enzymes combined with the endogenous chitinolytic enzymes produce by the plants. However, when control plants were trimmed, early flowering was observed compared with the controls that were not trimmed, which indicates that wound induced proteins such as chitinolytic enzymes affect the induction of flowering. As taught herein, the presence of endochitinase and chitobiosidase transgenes can significantly reduce developmental time by speeding up the process of flowering and fruiting of tomato, and increase the amount of fruits produced. One of the primary goals of all crop breeding programs is to increase the productivity of the plants. These two genes are directly associated with plant productivity.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of promoting early flowering in plants comprising:
providing a transgenic plant or plant seed transformed with a DNA molecule encoding a chitinolytic enzyme having chitinolytic activity and
growing the transgenic plant or transgenic plants produced from the transgenic plant seeds under conditions effective to promote early flowering in the plants.

2. A method according to claim 1, wherein a transgenic plant is provided.

3. A method according to claim 1, wherein a transgenic plant seed is provided.

4. A method according to claim 1, wherein the chitinolytic enzyme is selected from the group consisting of chitobiosidase, an endochitinase, and combinations thereof.

5. A method according to claim 4, wherein the chitinolytic enzyme is a Streptomyces albidoflavus chitinolytic enzyme.

6. A method according to claim 4, wherein the chitinolytic enzyme is chitobiosidase.

7. A method according to claim 6, wherein the chitobiosidase has an amino acid sequence comprising SEQ.ID.No. 1.

8. A method according to claim 6, wherein the chitobiosidase is encoded by a DNA molecule having a nucleotide sequence of SEQ.ID.No. 2 or hybridizing to a DNA molecule comprising a nucleotide sequence of SEQ.ID.No. 2 under stringent conditions characterized by a hybridization buffer comprising 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA at a temperature of 65° C.

9. A method according to claim 4, wherein the chitinolytic enzyme is an endochitinase.

10. A method according to claim 9, wherein the endochitinase has an amino acid sequence comprising SEQ.ID.No. 3.

11. A method according to claim 9, wherein the endochitinase is encoded by DNA molecule having a nucleotide sequence of SEQ.ID.No. 4 or hybridizing to a DNA molecule comprising a nucleotide sequence of SEQ.ID.No. 4 under stringent conditions characterized by a hybridization buffer comprising 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA, at a temperature of 65° C.

12. A method according to claim 1, wherein the plant is selected from the group consisting of Gramineae, Liliaceae, Iridaceae, Orchidaceae, Salicaceae, Ranunculaceae, Magnoliaceae, Cruciferae, Rosaceae, Leguminosae, Malvaceae, Umbelliferae, Labitatae, Solanceae, Cucurbitaceae, Compositae, and Rubiaceae.

13. A method of promoting yield from plants comprising:
providing a transgenic plant or plant seed transformed with a DNA molecule encoding a chitinolytic enzyme and
growing the transgenic plant or transgenic plants produced from the transgenic plant seeds under conditions effective to promote yield from the plants.

14. A method according to claim 13, wherein a transgenic plant is provided.

15. A method according to claim 13, wherein a transgenic plant seed is provided.

16. A method according to claim 13, wherein the chitinolytic enzyme is selected from the group consisting of a chitobiosidase, an endochitinase, and combinations thereof.

17. A method according to claim 16, wherein the chitinolytic enzyme is a Streptomyces albidoflavus chitinolytic enzyme.

18. A method according to claim 16, wherein the chitinolytic enzyme is chitobiosidase.

19. A method according to claim 18, wherein the chitobiosidase has an amino acid sequence comprising SEQ.ID.No. 1.

20. A method according to claim 18, wherein the chitobiosidase is encoded by a DNA molecule having a nucleotide sequence of SEQ.ID.No. 2 or hybridizing to a DNA molecule comprising a nucleotide sequence of SEQ.ID.No. 2 under stringent conditions characterized by a hybridization buffer comprising 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. Coli DNA, at a temperature of 65° C.

21. A method according to claim 16, wherein the chitinolytic enzyme is an endochitinase.

22. A method according to claim 21, wherein the endochitinase has an amino acid sequence comprising SEQ.ID.No. 3.

23. A method according to claim 21, wherein the endochitinase is encoded by a DNA molecule having a nucleotide sequence of SEQ.ID.No. 4 or hybridizing to a DNA molecule comprising a nucleotide sequence of SEQ.ID.No. 4 under stringent conditions characterized by a hybridization buffer comprising 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA at a temperature of 65° C.

24. A method according to claim 13, wherein the plant is selected from the group consisting of Gramineae, Liliaceae, Iridaceae, Orchidaceae, Salicaceae, Ranunculaceae, Magnoliaceae, Cruciferae, Rosaceae, Leguminosae, Malvaceae, Umbelliferae, Labitatae, Solanceae, Cucurbitaceae, Compositae, and Rubiaceae.

25. A method of reducing plant size comprising:
providing a transgenic plant or plant seed transformed with a DNA molecule encoding a chitinolytic enzyme and
growing the transgenic plant or transgenic plants produced from the transgenic plant seeds under conditions effective to reduce growth of the plants.

26. A method according to claim 25, wherein a transgenic plant is provided.

27. A method according to claim 25, wherein a transgenic plant seed is provided.

28. A method according to claim 25, wherein the chitinolytic enzyme is selected from the group consisting of a chitobiosidase, an endochitinase, and combinations thereof.

29. A method according to claim 28, wherein the chitinolytic enzyme is a Streptomyces albidoflavus chitinolytic enzyme.

30. A method according to claim 28, wherein the chitinolytic enzyme is chitobiosidase.

31. A method according to claim 30, wherein the chitobiosidase has an amino acid sequence comprising SEQ.ID.No. 1.

32. A method according to claim 30, wherein the chitobiosidase is encoded by a DNA molecule having a nucleotide sequence of SEQ.ID.No. 2 or hybridizing to a DNA molecule comprising a nucleotide sequence of SEQ.ID.No. 2 under stringent conditions characterized by a hybridization buffer comprising 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA at a temperature of 65° C.

33. A method according to claim 28, wherein the chitinolytic enzyme is an endochitinase.

34. A method according to claim 33, wherein the endochitinase has an amino acid sequence comprising SEQ.ID.No. 3.

35. A method according to claim 33, wherein the endochitinase is encoded by a DNA molecule having a nucleotide sequence of SEQ.ID.No. 4 or hybridizing to a DNA molecule comprising a nucleotide sequence of SEQ.ID.No. 4 under stringent conditions characterized by a hybridization buffer comprising 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA, at a temperature of 65° C.

36. A method according to claim 25, wherein the plant is selected from the group consisting of Gramineae, Liliaceae, Iridaceae, Orchidaceae, Salicaceae, Ranunculaceae, Magnoliaceae, Cruciferae, Rosaceae, Leguminosae, Malvaceae, Umbelliferae, Labitatae, Solanceae, Cucurbitaceae, Compositae, and Rubiaceae.

37. A method of promoting early flowering in plants comprising:
applying a chitinolytic enzyme having chitinolytic activity to a plant or plant seed under conditions effective to promote early flowering.

38. A method according to claim 37, wherein a transgenic plant is provided.

39. A method according to claim 37, wherein a transgenic plant seed is provided.

40. A method according to claim 37, wherein the chitinolytic enzyme is selected from the group consisting of chitobiosidase, endochitinase, and combinations thereof.

41. A method according to claim 40, wherein the chitinolytic enzyme is a Streptomyces albidoflavus chitinolytic enzyme.

42. A method according to claim 40, wherein the chitinolytic enzyme is chitobiosidase.

43. A method according to claim 42, wherein the chitobiosidase has an amino acid sequence comprising SEQ.ID.No. 1.

44. A method according to claim 40, wherein the chitinolytic enzyme is an endochitinase.

45. A method according to claim 44, wherein the endochitinase has an amino acid sequence comprising SEQ.ID.No. 3.

46. A method according to claim 37, wherein the plant is selected from the group consisting of Gramineae, Liliaceae, Iridaceae, Orchidaceae, Salicaceae, Ranunculaceae, Magnoliaceae, Cruciferae, Rosaceae, Leguminosae, Malvaceae, Umbelliferae, Labitatae, Solanceae, Cucurbitaceae, Compositae, and Rubiaceae.

47. A method of promoting yield from plants comprising:
applying a chitinolytic enzyme having chitinolytic activity to a plant or plant seed under conditions effective to promote yield.

48. A method according to claim 47, wherein a transgenic plant is provided.

49. A method according to claim 47, wherein a transgenic plant seed is provided.

50. A method according to claim 47, wherein the chitinolytic enzyme is selected from the group consisting of chitobiosidase, endochitinase, and combinations thereof.

51. A method according to claim 50, wherein the chitinolytic enzyme is a Streptomyces albidoflavus chitinolytic enzyme.

52. A method according to claim 50, wherein the chitinolytic enzyme is chitobiosidase.

53. A method according to claim 52, wherein the chitobiosidase has an amino acid sequence comprising SEQ.ID.No. 1.

54. A method according to claim 50, wherein the chitinolytic enzyme is an endochitinase.

55. A method according to claim 54, wherein the endochitinase has an amino acid sequence comprising SEQ.ID.No. 3.

56. A method according to claim 47, wherein the plant is selected from the group consisting of Gramineae, Liliaceae, Iridaceae, Orchidaceae, Salicaceae, Ranunculaceae, Magnoliaceae, Cruciferae, Rosaceae, Leguminosae, Malvaceae, Umbelliferae, Labitatae, Solanceae, Cucurbitaceae, Compositae, and Rubiaceae.

57. A method of reducing plant size comprising:
applying a chitinolytic enzyme having chitinolytic activity to a plant or plant seed under conditions effective to reduce plant size.

58. A method according to claim 57, wherein a transgenic plant is provided.

59. A method according to claim 57, wherein a transgenic plant seed is provided.

60. A method according to claim 57, wherein the chitinolytic enzyme is selected from the group consisting of chitobiosidase, endochitinase, and combinations thereof.

61. A method according to claim 60, wherein the chitinolytic enzyme is a Streptomyces albidoflavus chitinolytic enzyme.

62. A method according to claim 60, wherein the chitinolytic enzyme is chitobiosidase.

63. A method according to claim 62, wherein the chitobiosidase has an amino acid sequence comprising SEQ.ID.No. 1.

64. A method according to claim 60, wherein the chitinolytic enzyme is an endochitinase.

65. A method according to claim 64, wherein the endochitinase has an amino acid sequence comprising SEQ.ID.No. 3.

66. A method according to claim 57, wherein the plant is selected from the group consisting of Gramineae, Liliaceae, Iridaceae, Orchidaceae, Salicaceae, Ranunculaceae, Magnoliaceae, Cruciferae, Rosaceae, Leguminosae, Malvaceae, Umbelliferae, Labitatae, Solanceae, Cucurbitaceae, Compositae, and Rubiaceae.

67. A transgenic plant comprising:
a DNA molecule encoding a chitinolytic enzyme, wherein the DNA molecule is effective to promote early flowering.

68. A transgenic plant according to claim 67, wherein the chitinolytic enzyme is selected from the group consisting of chitobiosidase, endochitinase, and combinations thereof.

69. A transgenic plant comprising:
a DNA molecule encoding a chitinolytic enzyme, wherein the DNA molecule is effective to reduce plant growth.

70. A transgenic plant according to claim 69, wherein the chitinolytic enzyme is selected from the group consisting of chitobiosidase, endochitinase, and combinations thereof.

71. A transgenic plant comprising:
a DNA molecule encoding a chitinolytic enzyme, wherein the DNA molecule is effective to increase plant yield.

72. A transgenic plant according to claim 71, wherein the chitinolytic enzyme is selected from the group consisting of chitobiosidase, endochitinase, and combinations thereof.
